(19) United States
(12) Patent Application Publication
(10) Pub. No.: US 2023/0397880 A1
(43) Pub. Date: Dec. 14, 2023

(54) SYSTEMS AND METHODS FOR DETERMINING UNTREATED HEALTH-RELATED ISSUES

(71) Applicant: ResMed Pty Ltd, Bella Vista, NSW (AU)

(72) Inventors: Liam Holley, Sydney (AU); Ji Hae Jang, Gordon (AU)

(21) Appl. No.: 18/257,681

(22) PCT Filed: Dec. 14, 2021
(86) PCT No.: PCT/IB2021/061712

§ 371 Date: Jun. 15, 2023

Related U.S. Application Data
(60) Provisional application No. 63/125,663, filed on Dec. 15, 2020.
(60) Provisional application No. 63/241,297, filed on Sep. 7, 2021.

(51) Int. Cl. A61B 5/00; A61B 5/0205

(52) U.S. Cl. CPC A61B 5/4818; A61B 5/0205; A61B 5/7275; A61B 5/746; A61B 5/4842; A61B 5/02405

(57) ABSTRACT

First physiological data associated with a user during a first time period is received. The first physiological data is analyzed to determine (i) a first respiration rate for the first time period, (ii) a first plurality of sample heart rate values, and (iii) first heart rate variability parameters for the first time period. Second physiological data associated with the user during a second time period is received. The second physiological data is analyzed to determine (i) a second respiration rate for the second time period, (ii) a second plurality of sample heart rate values, and (iii) second heart rate variability parameters for the second time period, the second respiration rate being less than the first respiration rate. The percentage likelihood that the user has an untreated sleep disorder is determined based at least in part on the first heart rate variability parameters and the second heart rate variability parameters.

### CROSS-REFERENCE TO RELATED APPLICATIONS

This application claims the benefit of, and priority to, U.S. Provisional Patent Application No. 63/125,663 filed on Dec. 15, 2020, and U.S. Provisional Patent Application No. 63/241,297 filed on Sep. 7, 2021, each of which is hereby incorporated by reference herein in its entirety.

### TECHNICAL FIELD

The present disclosure relates generally to systems and methods for determining an untreated sleep disorder, or other health-related issues (e.g., issues that may impact sympathetic tone), and more particularly, to systems and methods for determining a percentage likelihood that a user has an untreated sleep disorder.

### BACKGROUND

Various systems exist for aiding users experiencing sleep apnea and related respiratory disorders. A range of respiratory disorders exist that can impact users. Certain disorders are characterized by particular events (e.g., apneas, hypopneas, hyperpneas, or any combination thereof). Examples of respiratory disorders include periodic limb movement disorder (PLMD), Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and Chest wall disorders. Thus, a need exists for systems and methods for identifying an individual with untreated health-related issues, such as respiratory disorders.

These disorders are often treated using a respiratory therapy system. However, some users find such systems to be uncomfortable, difficult to use, expensive, aesthetically unappealing and/or fail to perceive the benefits associated with using the system. As a result, some users will elect not to begin using the respiratory therapy system or discontinue use of the respiratory therapy system absent a demonstration of the severity of their symptoms when respiratory therapy treatment is not used. In addition, some individuals not using the respiratory therapy system may not realize that they suffer from one or more sleep-related and/or respiratory-related disorders. Furthermore, some users may only suffer from certain symptoms when sleeping in a specific body position.

The present disclosure is directed to solving these and other problems.

### SUMMARY

According to some implementations of the present disclosure, a method for determining a percentage likelihood that a user has an untreated sleep disorder is disclosed as follows. First physiological data associated with the user during a first time period is received. The first physiological data is analyzed to determine (i) a first respiration rate for the first time period, (ii) a first plurality of sample heart rate values, and (iii) first heart rate variability parameters for the first time period. Second physiological data associated with the user during a second time period is received. The second physiological data is analyzed to determine (i) a second respiration rate for the second time period, (ii) a second plurality of sample heart rate values, and (iii) second heart rate variability parameters for the second time period, the second respiration rate being less than the first respiration rate. The percentage likelihood that the user has an untreated sleep disorder is determined based at least in part on the first heart rate variability parameters and the second heart rate variability parameters.

According to some implementations of the present disclosure, a system for determining a percentage likelihood that a user has an untreated sleep disorder is disclosed as follows. The system includes a control system configured to implement the method disclosed above.

According to some implementations of the present disclosure, a method is disclosed as follows. Positional data associated with a user is received. The received positional data is analyzed to determine a body position of the user. Based at least in part on the determined body position of the user, the user is caused to change body position.

According to some implementations of the present disclosure, a system for monitoring a body position of a user is disclosed as follows. The system includes a control system configured to implement the method disclosed above.

According to some implementations of the present disclosure, a system includes a control system and a memory. The control system includes one or more processors. The memory has stored thereon machine readable instructions. The control system is coupled to the memory. Any one of the methods disclosed herein is implemented when the machine executable instructions in the memory are executed by at least one of the one or more processors of the control system.

According to some implementations of the present disclosure, a computer program product comprising instructions which, when executed by a computer, cause the computer to carry out any one of the methods disclosed herein. In some implementations, the computer program product is a non-transitory computer readable medium.

According to some implementations of the present disclosure, a wearable device includes a treatment device and a strap coupled to the treatment device. The treatment device includes a concave surface and a convex surface. The concave surface is configured to contact a back of a head of a user while sleeping. The treatment device is bi-stable on the convex surface, such that the treatment device is stable when positioned on either side of the convex surface, and unstable when positioned about a vertex of the convex surface. The strap is configured to be worn around the head of the user to secure the treatment device to the back of the head of the user.

According to some implementations of the present disclosure, a wearable device includes a treatment device and a strap coupled to the treatment device. The treatment device includes a concave surface and an opposite surface. The concave surface is configured to contact a back of a head of a user while sleeping. The treatment device is weighted to be bi-stable on the opposite surface, such that the treatment device is stable when positioned on either side of the opposite surface, and unstable when positioned about a center of the opposite surface. The strap is configured to be worn around the head of the user to secure the treatment device to the back of the head of the user.

According to some implementations of the present disclosure, a method provides generating physiological data associated with the user via any of the treatment devices disclosed above. The method further provides determining whether the user has sleep apnea based at least in part on the generated physiological data associated with the user.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

The foregoing and other advantages of the present disclosure will become apparent upon reading the following detailed description and upon reference to the drawings.

FIG. 1 is a functional block diagram of a system for monitoring a user, according to some implementations of the present disclosure.

FIG. 2 illustrates a flow diagram for a method for determining a percentage likelihood that a user has an untreated sleep disorder, according to some implementations of the present disclosure.

FIG. 3 illustrates a mobile device having at least a portion of the system of FIG. 1, according to some implementations of the present disclosure.

FIG. 4 is a perspective view of a user and the mobile device of FIG. 3, according to some implementations of the present disclosure.

FIG. 5 illustrates physiological data received during a first time period, according to some implementations of the present disclosure.

FIG. 6 illustrates physiological data received during a second time period, according to some implementations of the present disclosure.

FIG. 7 illustrates physiological data associated with a user without a sleep disorder, according to some implementations of the present disclosure.

FIG. 8 illustrates physiological data associated with a user having untreated OSA, according to some implementations of the present disclosure.

FIG. 9 illustrates a displayed indication to a user who is likely to have untreated OSA, according to some implementations of the present disclosure.

FIG. 10 illustrates a displayed indication to a user who is unlikely to have untreated OSA, according to some implementations of the present disclosure.

FIG. 11 a perspective view of a user wearing a mobile device having at least a portion of the system of FIG. 1 and in a supine body position, according to some implementations of the present disclosure.

FIG. 12 a perspective view of the user of FIG. 11 in a side body position, according to some implementations of the present disclosure.

FIG. 13 illustrates a flow diagram for a method for monitoring a body position of a user, according to some implementations of the present disclosure.

FIG. 14 is a perspective view of at least a portion of the system of FIG. 1, a user, and a bed partner, according to some implementations of the present disclosure.

FIG. 15 is a top perspective view of at least a portion of the system of FIG. 1 and a user wearing a treatment device, according to some implementations of the present disclosure.

FIG. 16 is a side view of the user wearing the treatment device of FIG. 15, according to some implementations of the present disclosure.

FIG. 17A illustrates that the user wearing the treatment device of FIG. 15 moves from facing upright to facing left, according to some implementations of the present disclosure.

FIG. 17B illustrates that the user wearing the treatment device of FIG. 15 moves from facing upright to facing right, according to some implementations of the present disclosure.

FIG. 18A is a top perspective view of the user wearing the treatment device of FIG. 15 and facing left, according to some implementations of the present disclosure.

FIG. 18B is a side view of the user wearing the treatment device of FIG. 15 and facing left, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA), and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hyper tension, diabetes, stroke, insomnia, and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in International Pub. No. WO 2008/138040 and U.S. Pat. No. 9,358,353, assigned to ResMed Ltd., the disclosure of each of which is hereby incorporated by reference herein in their entireties.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

Various embodiments of the present disclosure are directed to a wearable device that aids in keeping a user's head on the side to help address positional sleep apnea. The wearable device can be passive and/or powered to aid the user. The wearable device can be used independently or in conjunction with a respiratory therapy system. The wearable device may include and/or be in communications with one or more sensors to monitor a body position and/or a head position of the user, and/or other physiological data (e.g., sleep apnea, heart rate, heart rate variability, etc.). The measured data may be used as an input to a connected respiratory therapy system and/or another wearable device. Additionally or alternatively, the measured data may be used as feedback to the user via a connected smart device after one or more sleep sessions wearing the wearable device.

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

The present disclosure relates to systems and methods that utilize a device to obtain cardiac signals from a user to determine one or more heart rate variability parameters, which may be analyzed to determine whether the user is likely to have a sleep disorder (e.g., OSA). The device may include an accelerometer, and/or a heart rate/pulse sensor (e.g., a pulse oximeter, ECG). The device may also provide prompts to the user to go through a deep breathing exercise whilst measuring the signals. In some implementations, the output is an indication of risk of OSA for the user.

The present disclosure also relates to systems and methods that utilize a device that measures and/or records various signals (e.g. positional signals, cardiac signals, breathing signals) to provide positional therapy (e.g. by buzzing or prompting the user to roll to their side when the user is supine, and/or when detecting apnea events). The systems may include a coupling mechanism (e.g., strap) that holds the device in place, yet is comfortable enough for the user to fall asleep with.

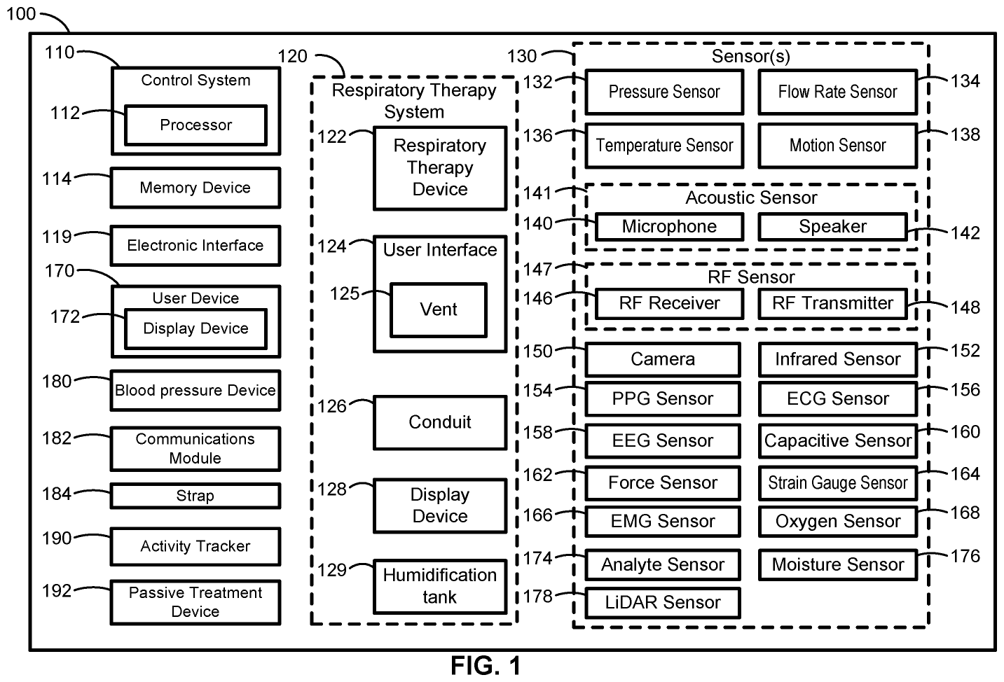

Referring to FIG. 1, a system 100, according to some implementations of the present disclosure, is illustrated. The system 100 may be for providing a variety of different sensors related to a user's use of a mobile device, among other uses. The system 100 includes a control system 110, a memory device 114, an electronic interface 119, one or more sensors 130, and one or more mobile devices 170. In some implementations, the system 100 further includes a strap 184 for coupling the one or more mobile devices 170 to the user. The system 100 can be used to identify an untreated health-related issues (e.g., any disease or condition that increases sympathetic activity, such as sleep disorder, COPD, CVD, acute respiratory distress, somatic syndromes) and/or a body position of a user, as disclosed in further detail herein.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the mobile device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the mobile device 170, within a housing of one or more of the sensors 130, or both. Like the control system 110, the memory device 114 can be centralized (within one such housing) or decentralized (within two or more of such housings, which are physically distinct).

In some implementations, the memory device 114 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof.

The medical information can include, for example, information indicative of one or more medical conditions associated with the user, medication usage by the user, or both. The medical information data can further include a multiple sleep latency test (MSLT) test result or score, a Pittsburgh Sleep Quality Index (PSQI) score or value, an Epworth Sleepiness Score (ESS), and/or the results of other patient surveys. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The medical information data can include results from one or more of a polysomnography (PSG) test, a CPAP titration, or a home sleep test (HST), respiratory therapy system settings from one or more sleep sessions, sleep related respiratory events from one or more sleep sessions, or any combination thereof. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof. In some implementations, the memory device 114 stores media content that can be displayed on the display device 172. In some implementations, a short, and/or long-term history of information may be stored and analyzed, such that trend data may be displayed or acted upon. In some implementations, trend data may be used as a metric to monitor for improvement or deterioration of a condition, for example, an increase in heart rate variability after the onset of a particular type of therapy, such as CPAP, or positional OSA therapy, may indicate that the therapy is working. As a result, a message may be generated to inform a patient or clinician that they treatment is working and encourage them the persist with therapy. Conversely, if the data indicate either no change or a deterioration in a condition, a message may be generated to indicate to the patient or a clinician that may prompt them to consider alternative therapies.

The electronic interface 119 is configured to receive data (e.g., physiological data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a Wi-Fi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the mobile device 170. In other implementations, the electronic interface 119 is coupled to or integrated (e.g., in a housing) with the control system 110 and/or the memory device 114.

Still referring to FIG. 1, in some implementations, the system 100 further optionally includes a respiratory therapy system 120, a blood pressure device 180, an activity tracker 190, or any combination thereof. The respiratory therapy system 120 can include a respiratory pressure therapy device (RPT) 122 (referred to herein as respiratory therapy device 122), a user interface 124 (also called a ‘mask’), a conduit 126 (also referred to as a tube or an air circuit), a display device 128, a humidification tank 129, or any combination thereof.

In some implementations, the control system 110, the memory device 114, the display device 128, one or more of the sensors 130, and the humidification tank 129 are part of the respiratory therapy device 122. Respiratory pressure therapy refers to the application of a supply of air to an entrance of the user's airways at a controlled target pressure that is nominally positive with respect to atmosphere throughout the user's respiratory cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 has a blower motor (not shown) that is generally used to generate pressurized air that is delivered to the user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 liters/minute and about 150 liters/minute, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory therapy device 122, the user interface 124, and the conduit 126 form an air pathway fluidly coupled with an airway of the user. The pressurized air also increases the user's oxygen intake during sleep. Depending upon the therapy to be applied, the user interface 124 may form a seal, for example, with a region or portion of the user's face, to facilitate the delivery of air at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

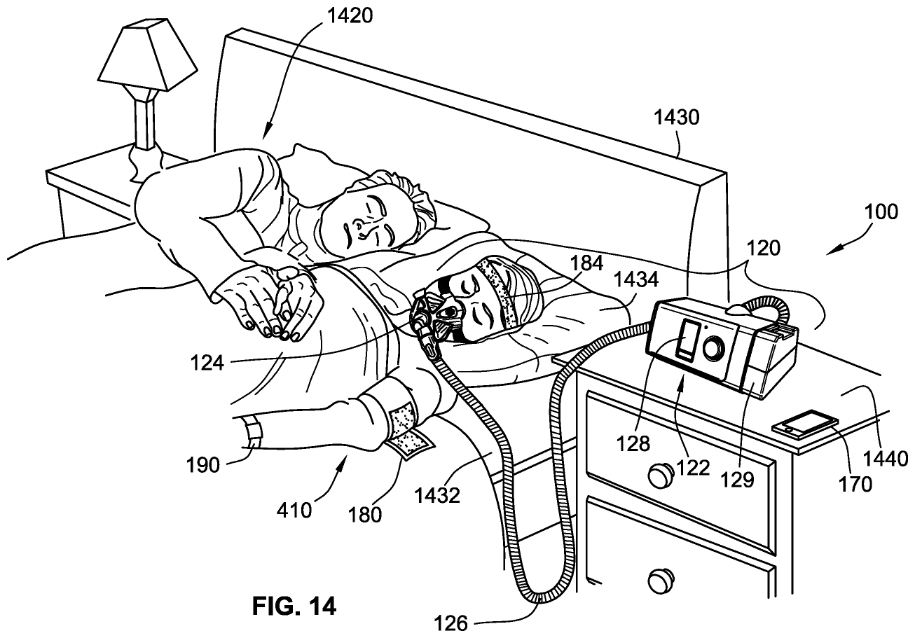

As shown in FIG. 14, in some implementations, the user interface 124 is a facial mask (e.g. a full face mask) that covers the nose and mouth of the user 410. Alternatively, the user interface 124 can be a nasal mask that provides air to the nose of the user 410 or a nasal pillow mask that delivers air directly to the nostrils of the user 410. The user interface 124 can include a plurality of straps forming, for example, a headgear for aiding in positioning and/or stabilizing the interface on a portion of the user 410 (e.g., the face) and a conformal cushion (e.g., silicone, plastic, foam, etc.) that aids in providing an air-tight seal between the user interface 124 and the user 410. The user interface 124 can also include one or more vents 125 for permitting the escape of carbon dioxide and other gases exhaled by the user 410. In other implementations, the user interface 124 includes a mouthpiece (e.g., a night guard mouthpiece molded to conform to the teeth of the user 410, a mandibular repositioning device, etc.).

The conduit 126 (also referred to as an air circuit or tube) allows the flow of air between two components of a respiratory therapy system 120, such as the respiratory therapy device 122 and the user interface 124. In some implementations, there can be separate limbs of the conduit 126 for inhalation and exhalation. In other implementations, a single limb conduit is used for both inhalation and exhalation.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score and/or a therapy score, also referred to as a myAir™ score, such as described in International Pub. No. WO 2016/061629 and U.S. Patent Pub. No. 2017/0311879, each of which is hereby incorporated by reference herein in its entirety; the current date/time; personal information for the user 410; questions seeking feedback from the user and/or advice to the user; etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include one or more vents (not shown) and a heater to heat the water in the humidification tank 129 in order to humidify the pressurized air provided to the user 410. Additionally, in some implementations, the conduit 126 can also include a heating element (e.g., coupled to and/or embedded in the conduit 126) that heats the pressurized air delivered to the user 410. The humidification tank 129 can be fluidly coupled to a water vapor inlet of the air pathway and deliver water vapor into the air pathway via the water vapor inlet, or can be formed in-line with the air pathway as part of the air pathway itself. In some implementations, the humidification tank 129 may not include the reservoir of water and thus waterless.

In some implementations, the system 100 can be used to deliver at least a portion of a substance from the receptacle (not shown) to the air pathway of the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) a combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined amount of pressurized air (e.g., determined by a sleep physician) to the user 410. The APAP system automatically varies the pressurized air delivered to the user 410 based on, for example, respiration data associated with the user 410. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

The one or more sensors 130 (or transducers) of the system 100 include a pressure sensor 132, a flow rate sensor 134, a temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178 more generally, the one or more sensors 130 can include any combination and any number of each of the sensors described and/or shown herein.

As described herein, the system 100 generally can be used to generate physiological data associated with a user during one or more time periods. The physiological data can be analyzed to generate one or more heart rate parameters (such as the beat by beat rate), or hear rate variability parameters, respiratory parameters (such as respiratory rate or amplitude, respiratory effort or a proxy (e.g., respiratory muscle activity for any of the respiratory muscles)), sleep-related parameters, and/or any other parameter, measurement, etc. related to the user during the time period. The one or more heart rate variability parameters that can be determined for the user during the one or more time periods include, for example, a plurality of heart rates, a maximum heart rate, a minimum heart rate, a heart rate range, an average heart rate, a median heart rate, a standard deviation of heart rates. Additionally or alternatively, in some implementations, the one or more heart rate variability parameters can include one or more short-term (e.g., about or less than 5 minutes) heart rate variability parameters, long-term (e.g., more than 5 minutes, such as 24 hours) heart rate variability parameters, or both. Additionally or alternatively, in some implementations, the one or more heart rate variability parameters can include any statistical metrics, such as mean, standard deviation, etc., derived from time intervals between features in a measured signal, such as peaks in an ECG or an accelerometer signal. In some implementations, the one or more heart rate variability parameters may include signal power or peak values in a specified frequency bandwidth, for example, ultra-low frequency bandwidth including frequencies less than 0.003 Hz. Other frequency ranges may include 0.003 to 0.04 Hz, and 0.04 to 0.15 Hz, and 0.15 Hz to 0.4 Hz. EEG activity, EMG activity, or any combination thereof. The one or more sleep-related parameters that can be determined for the user during the one or more time periods include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of a respiratory device, a heart rate, a heart rate variability, movement of the user, temperature, EEG activity, EMG activity, arousal, snoring, choking, coughing, whistling, wheezing, or any combination thereof.

The one or more sensors 130 can be used to generate, for example, physiological data, positional data, or both. In some implementations, the physiological data generated by one or more of the sensors 130 can be used by the control system 110 to determine a sleep-wake signal associated with the user during a sleep session and one or more sleep-related parameters. The sleep-wake signal can be indicative of one or more sleep states, including sleep, wakefulness, relaxed wakefulness, micro-awakenings, or distinct sleep stages such as a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 130, are described in, for example, U.S. Pat. No. 10,492,720, U.S. Patent Pub. No. 2014/0088373, International Pub. No. WO 2017/132726, International Pub. No. WO 2019/122413, International Pub. No. WO 2019/122414, and U.S. Patent Pub. No. 2020/0383580, each of which is hereby incorporated by reference herein in its entirety.

The sleep-wake signal can also be timestamped to determine a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory device, or any combination thereof during the sleep session. The event(s) can include snoring, apneas, central apneas, obstructive apneas, mixed apneas, hypopneas, a mouth leak, a mask leak, a restless leg, a sleeping disorder, choking, an increased heart rate, a heart rate variation, labored breathing, an asthma attack, an epileptic episode, a seizure, a fever, a cough, a series of coughs (e.g., mucus producing or not), a sneeze, a snore, a gasp, an episode of respiratory insufficiency, the presence of an illness such as the common cold or the flu, or any combination thereof. The one or more sleep-related parameters that can be determined for the user during the sleep session based on the sleep-wake signal include, for example, sleep quality metrics such as a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

The physiological data generated by the one or more sensors 130 can also be used to determine a respiration signal associated with a user during the one or more time periods and/or a sleep session. The respiration signal is generally indicative of respiration or breathing of the user during the one or more time periods and/or the sleep session. The respiration signal can be indicative of and/or analyzed to determine (e.g., using the control system 110) one or more sleep-related parameters, such as, for example, a respiration rate, a respiration rate variability, an inspiration amplitude, an expiration amplitude, an inspiration-expiration ratio, an inspiration and/or expiration duration, an occurrence of one or more events, a number of events per hour, a pattern of events, a sleep state, a sleep stage, an apnea-hypopnea index (AHI), pressure settings of the respiratory device, or any combination thereof. The one or more events can include snoring, apneas, central apneas, obstructive apneas, mixed apneas, hypopneas, a mouth leak, a mask leak, a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 130, or from other types of data.

Generally, the sleep session includes any point in time after the user has laid or sat down in the bed (or another area or object on which they intend to sleep). The sleep session can thus include time periods (i) when the user is using a respiratory therapy system but before the user attempts to fall asleep (for example when the user lays in the bed reading a book); (ii) when the user begins trying to fall asleep but is still awake; (iii) when the user is in a light sleep (also referred to as stage 1 and stage 2 of non-rapid eye movement (NREM) sleep); (iv) when the user is in a deep sleep (also referred to as slow-wave sleep, SWS, or stage 3 of NREM sleep); (v) when the user is in rapid eye movement (REM) sleep; (vi) when the user is periodically awake between light sleep, deep sleep, or REM sleep; or (vii) when the user wakes up and does not fall back asleep. The sleep session is generally defined as ending once the user, turns off the respiratory device, and/or gets out of bed. In some implementations, the sleep session can include additional periods of time, or can be limited to only some of the above-disclosed time periods.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user and/or ambient pressure. In such implementations, the pressure sensor 132 can be coupled to or integrated in the mobile device 170. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of a user.

The flow rate sensor 134 outputs flow rate data that can be stored in the memory device 114 and/or analyzed by the processor 112 of the control system 110. Examples of flow rate sensors (such as, for example, the flow rate sensor 134) are described in International Pub. No. No. WO 2012/012835 and U.S. Pat. No. 10,328,219, each of which is hereby incorporated by reference herein in its entirety. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In one example, the pressure sensor 132 can be used to determine a blood pressure of a user.

The temperature sensor 136 outputs temperature data that can be stored in the memory device 114 and/or analyzed by the processor 112 of the control system 110. In some implementations, the temperature sensor 136 generates temperature data indicative of a core body temperature of the user, a skin temperature of the user, a temperature of the air flowing from the respiratory therapy device and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user during the one or more time periods, and/or a body orientation of the user. In some implementations, the motion sensor 138 can be used to detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state of the user; for example, via a respiratory movement of the user. In some implementations, the motion data from the motion sensor 138 can be used in conjunction with additional data from another sensor 130 to determine the sleep state of the user.

The microphone 140 outputs sound and/or audio data that can be stored in the memory device 114 and/or analyzed by the processor 112 of the control system 110. The audio data generated by the microphone 140 is reproducible as one or more sound(s) during a sleep session (e.g., sounds from the user). The audio data form the microphone 140 can also be used to identify (e.g., using the control system 110) an event experienced by the user during the sleep session, as described in further detail herein. The microphone 140 can be coupled to or integrated in the respiratory therapy device 122, the user interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves. In one or more implementations, the sound waves can be audible to a user of the system 100 or inaudible to the user of the system (e.g., ultrasonic sound waves). The speaker 142 can be used, for example, as an alarm clock or to play an alert or message to the user (e.g., in response to an identified body position and/or a change in body position). In some implementations, the speaker 142 can be used to communicate the audio data generated by the microphone 140 to the user. The speaker 142 can be coupled to or integrated in the mobile device 170.

The microphone 140 and the speaker 142 can be used as separate devices. In some implementations, the microphone 140 and the speaker 142 can be combined into an acoustic sensor 141, as described in, for example, WO 2018/050913 and WO 2020/104465, each of which is hereby incorporated by reference herein in its entirety. In such implementations, the speaker 142 generates or emits sound waves at a predetermined interval and the microphone 140 detects the reflections of the emitted sound waves from the speaker 142. In one or more implementations, the sound waves generated or emitted by the speaker 142 can have a frequency that is not audible to the human ear (e.g., below 20 Hz or above around 18 kHz) so as not to disturb the user. Based at least in part on the data from the microphone 140 and/or the speaker 142, the control system 110 can determine a location of the user, one or more of the heart rate variability parameters, and/or one or more of the sleep-related parameters (e.g., an identified body position and/or a change in body position) described in herein such as, for example, a respiration signal, a respiration rate, an inspiration amplitude, an expiration amplitude, an inspiration-expiration ratio, a number of events per hour, a pattern of events, a sleep state, pressure settings of the respiratory device, or any combination thereof. In this context, a sonar sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO2018/050913 and WO 2020/104465 mentioned above.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location and/or a body position of the user, one or more heart rate variability parameters, and/or one or more of the sleep-related parameters described herein. An RF receiver (either the RF receiver 146 and the RF transmitter 148 or another RF pair) can also be used for wireless communication between the control system 110, the one or more sensors 130, the mobile device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g. a RADAR sensor). In some such implementations, the RF sensor 147 includes a control circuit. The specific format of the RF communication could be Wi-Fi, Bluetooth, or etc.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the heart rate variability parameters and/or one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to identify a location and/or a body position of the user, to determine chest movement of the user, to determine air flow of the mouth and/or nose of the user, to determine a time when the user enters the bed, and to determine a time when the user exits the bed. The camera 150 can also be used to track eye movements, pupil dilation (if one or both of the user's eyes are open), blink rate, or any changes during REM sleep. In some implementations, the camera 150 includes a wide angle lens or a fish eye lens.

The infrared (IR) sensor 152 outputs infrared image data reproducible as one or more infrared images (e.g., still images, video images, or both) that can be stored in the memory device 114. The infrared data from the IR sensor 152 can be used to determine one or more of the heart rate variability parameters and/or one or more sleep-related parameters, including a temperature of the user and/or movement of the user. The IR sensor 152 can also be used in conjunction with the camera 150 when measuring the presence, location, and/or movement of the user. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user that can be used to determine one or more of the heart rate variability parameters and/or one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user, embedded in and/or coupled to the user interface 124 and/or its associated headgear (e.g., straps, etc.), embedded in clothing and/or fabric that is worn by the user, embedded in and/or coupled to the mobile device 170.

The ECG sensor 156 outputs physiological data associated with electrical activity of the heart of the user. In some implementations, the ECG sensor 156 includes one or more electrodes that are positioned on or around a portion of the user during the one or more time periods and/or the sleep session. The physiological data from the ECG sensor 156 can be used, for example, to determine one or more of the heart rate variability parameters and/or one or more of the sleep-related parameters described herein.

The EEG sensor 158 outputs physiological data associated with electrical activity of the brain of the user. In some implementations, the EEG sensor 158 includes one or more electrodes that are positioned on or around the scalp of the user during the one or more time periods and/or the sleep session. The physiological data from the EEG sensor 158 can be used, for example, to determine a sleep state of the user at any given time during the one or more time periods and/or the sleep session. In some implementations, the EEG sensor 158 can be integrated in the mobile device 170 and/or a separate headgear.

The capacitive sensor 160, the force sensor 162, and the strain gauge sensor 164 output data that can be stored in the memory device 114 and used by the control system 110 to determine one or more of the heart rate variability parameters and/or one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user. The data output by the analyte sensor 174 can be stored in the memory device 114 and used by the control system 110 to determine the identity and concentration of any analytes in the breath of the user. In some implementations, the analyte sensor 174 is positioned near a mouth of the user to detect analytes in breath exhaled from the user's mouth. For example, when the user interface 124 is a full face mask that covers the nose and mouth of the user, the analyte sensor 174 can be positioned within the full face mask to monitor the user's mouth breathing. In other implementations, such as when the user interface 124 is a nasal mask or a nasal pillow mask, the analyte sensor 174 can be positioned near the nose of the user to detect analytes in breath exhaled through the user's nose. In still other implementations, the analyte sensor 174 can be positioned near the user's mouth when the user interface 124 is a nasal mask or a nasal pillow mask. In this implementation, the analyte sensor 174 can be used to detect whether any air is inadvertently leaking from the user's mouth. In some implementations, the analyte sensor 174 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 174 positioned near the mouth of the user or within the full face mask (in implementations where the user interface 124 is a full face mask) detects the presence of an analyte, the control system 110 can use this data as an indication that the user is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user's face, near the connection between the conduit 126 and the user interface 124, near the connection between the conduit 126 and the respiratory therapy device 122, etc.). Thus, in some implementations, the moisture sensor 176 can be coupled to or integrated in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the control system 110, the respiratory therapy device 122, the user interface 124, the conduit 126, the humidification tank 129, the control system 110, the user device 170, the activity tracker 190, or any combination thereof. For example, the acoustic sensor 141 and/or the RF sensor 147 can be integrated in and/or coupled to the mobile device 170. In some implementations, at least one of the one or more sensors 130 is not physically and/or communicatively coupled to the control system 110 or the mobile device 170, and is positioned generally adjacent to the user during the one or more time periods and/or sleep session (e.g., positioned on or in contact with a portion of the user, worn by the user, coupled to or positioned on the nightstand, coupled to the mattress, coupled to the ceiling, etc.).

The data from the one or more sensors 130 can be analyzed to determine one or more of the heart rate variability parameters and/or one or more sleep-related parameters. In some implementations, the one or more sleep-related parameters can include a sleep score, such as the ones described in International Publication No. WO 2015/006364 and U.S. Pat. No. 10,376,670, each of which is hereby incorporated by reference herein in its entirety. The one or more sleep-related parameters can include any number of sleep-related parameters (e.g., 1 sleep-related parameter, 2 sleep-related parameters, 5 sleep-related parameters, 50 sleep-related parameters, etc.). In some implementations, the one or more sleep-related parameters can include a heart rate, a heart rate variability, a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, an intentional mask leak, an unintentional mask leak, a mouth leak, a cough, a restless leg, a sleeping disorder, choking, an increased heart rate, labored breathing, an asthma attack, an epileptic episode, a seizure, increased or decreased blood pressure, any cardiac arrhythmia (such as atrial fibrillation), a COPD exacerbation, a rhinitis exacerbation, a syncope, or any combination thereof. Many of these parameters are physiological parameters, although some of the parameters can be considered to be non-physiological parameters. Non-physiological parameters can also include operational parameters of the respiratory system, including flow rate, pressure, humidity of the pressurized air, speed of motor, etc. Other types of physiological and non-physiological parameters can also be determined, either from the data from the one or more sensors 130, or from other types of data.

The mobile device 170 includes a display device 172. The mobile device 170 can be, for example, a mobile device such as a smart phone, a tablet, a gaming console, a smart watch, a laptop, or the like. Alternatively, the mobile device 170 can be an external sensing system, a television (e.g., a smart television) or another smart home device (e.g., a smart speaker(s) such as Google Home, Amazon Echo, Alexa etc.). In some implementations, the mobile device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the mobile device 170. In some implementations, one or more mobile devices can be used by and/or included in the system 100.

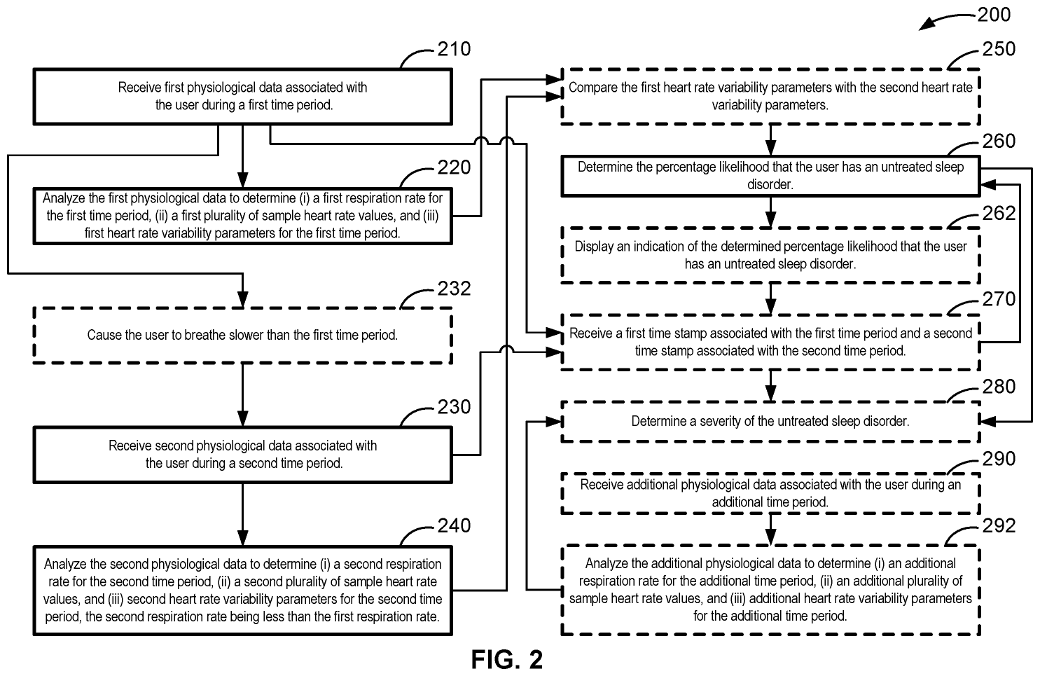

Referring to FIG. 2, a flow diagram for a method 200 for determining a percentage likelihood that a user has an untreated sleep disorder is disclosed. Additionally or alternatively, in some implementations, the output is any scale proportional to the likelihood that the user has an untreated sleep disorder. Additionally or alternatively, in some implementations, the output is simply a yes/no classification of untreated sleep disorder. For example, in some implementations, the output is low, medium, or high. As another example, in some implementations, the output is low, or high. As a further example, in some implementations, the output is yes or no. As yet another example, in some implementations, the output is a numeral score. At step 210, first physiological data associated with the user during a first time period is received. At step 220, the first physiological data received at step 210 is analyzed to determine (i) a first respiration rate for the first time period, (ii) a first plurality of sample heart rate values, and (iii) first heart rate variability parameters for the first time period. In some implementations, the first heart rate variability parameters for the first time period include a maximum heart rate for the first time period, a minimum heart rate for the first time period, a heart rate range defined by the maximum heart rate and the minimum heart rate for the first time period, an average heart rate for the first time period, a median heart rate for the first time period, a standard deviation of heart rates for the first time period, or any combination thereof.

Additionally or alternatively, in some implementations, the heart rate variability parameters include any parameters that are associated with the variability of heart rates. For example, in some implementations, the heart rate variability parameters may include (i) SDNN: the standard deviation of NN intervals, which is calculated over a 24-hour period; SDANN, the standard deviation of the average NN intervals calculated over short periods, such as 5 minutes; SDANN is therefore a measure of changes in heart rate due to cycles longer than 5 minutes; and SDNN reflects all the cyclic components responsible for variability in the period of recording, therefore it represents total variability; (ii) RMS SD (“root mean square of successive differences”): the square root of the mean of the squares of the successive differences between adjacent NNs; (iii) SDSD (“standard deviation of successive differences”): the standard deviation of the successive differences between adjacent NNs; (iv) NN50: the number of pairs of successive NNs that differ by more than 50 ms; (v) pNN50: the proportion of NN50 divided by total number of NNs; (vi) NN20: the number of pairs of successive NNs that differ by more than 20 ms; (vii) pNN20: the proportion of NN20 divided by total number of NNs; (viii) EBC (“estimated breath cycle”): the range (max-min) within a moving window of a given time duration within the study period; the windows can move in a self-overlapping way or be strictly distinct (sequential) windows; EBC is often provided in data acquisition scenarios where HRV feedback in real time is a primary goal; and EBC derived from PPG over 10-second and 16-second sequential and overlapping windows has been shown to correlate highly with SDNN; or (ix) any combination thereof.

At step 230, second physiological data associated with the user during a second time period is received. At step 240, the second physiological data received at step 230 is analyzed to determine (i) a second respiration rate for the second time period, (ii) a second plurality of sample heart rate values, and (iii) second heart rate variability parameters for the second time period, the second respiration rate being less than the first respiration rate. In some implementations, the second heart rate variability parameters for the second time period include a maximum heart rate for the second time period, a minimum heart rate for the second time period, a heart rate range defined by the maximum heart rate and the minimum heart rate for the second time period, an average heart rate for the second time period, a median heart rate for the second time period, a standard deviation of heart rates for the second time period, or any combination thereof. In some implementations, the second physiological data associated with the user during the second time period is obtained and/or extracted from data collected over long periods (e.g., without prompting the user to breathe slowly).

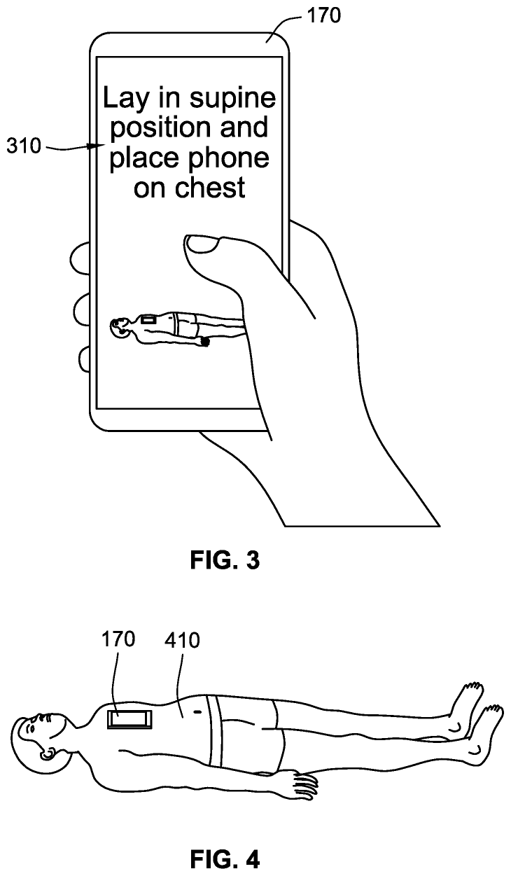

In some implementations, at step 232 and prior to receiving the second physiological data, the user is caused to breathe slower than they did in step 210 (e.g., slower than the first respiration rate determined at step 220). For example, referring briefly to FIGS. 3-4, a visual indication 310 (e.g., in the form of text, video, graphical indicator, screen brightness, an LED indication, a flash variation, etc.) may be displayed to a user 410 via the mobile device 170. Additionally or alternatively, an audio indication may be played to the user 410. The user 410 may be instructed to lay in supine position and place the mobile device 170 on their chest.

In some implementations, the mobile device 170 may be placed on and/or secured to the user 410 by any suitable means, such as via a strap, a clip, an elastic, a temporary adhesive, etc. For example, in some implementations, the mobile device 170 may be a foldable phone with a smaller footprint than a traditional mobile phone. The foldable phone can be more comfortable to fall asleep wearing. In some such implementations, the foldable phone may be clipped onto a front pocket of a T-shirt that the user is wearing. As another example, in some implementations, the mobile device 170 may be a smartwatch. In some such implementations, the smartwatch may be placed in the front pocket of a T-shirt that the user is wearing, with or without the watchband. Additionally or alternatively, in some such implementations, the smartwatch may be converted as part of a necklace to be worn by the user.

Additionally or alternatively, the user maybe instructed to stay still and relax (e.g., via the visual indication 310, via vibration of the mobile device 170, etc.). Additionally or alternatively, the user may be instructed to do any or all of, take a deep breath, relax, think of one or more pleasant thoughts or experiences, clear their mind, eat or drink something (such as a glass of water, or a light snack). Additionally or alternatively, the user may be exposed to a stimulus, such as an audio and/or video stimulus designed to relax the user. For example, the stimulus might take a form that is commonly considered to be relaxing, such as an audio and or visual representation of a natural environment, or a calm or pleasant story.

In some implementations, the physiological data may include sleep state, cardiac arrhythmia, nasal cannula, pulse oxygen, actigraphy, or any combination thereof. Additionally or alternatively, in some implementations, the physiological data may be analyzed to detect sleep state, cardiac arrhythmia, nasal cannula, pulse oxygen, actigraphy, or any combination thereof. For example, a determination of sleep state can be augmented by analyzing the actigraphy and the heart rate.

In some implementations, the first physiological data may be received (step 210 of FIG. 2) as the baseline for the user 410, then the user is guided to slow their breathing so that the second physiological data may be received (step 230 of FIG. 2). In some implementations, after the first physiological data and/or the second physiological data are received, and/or after a predetermined time period (e.g., one minute, two minutes, three minutes, etc.), the user may be notified that the test is complete and/or the results are available to be displayed. In some implementations, a feedback loop is included, where the user may be instructed to move the phone 170 if the signal is weak for collecting the physiological data.

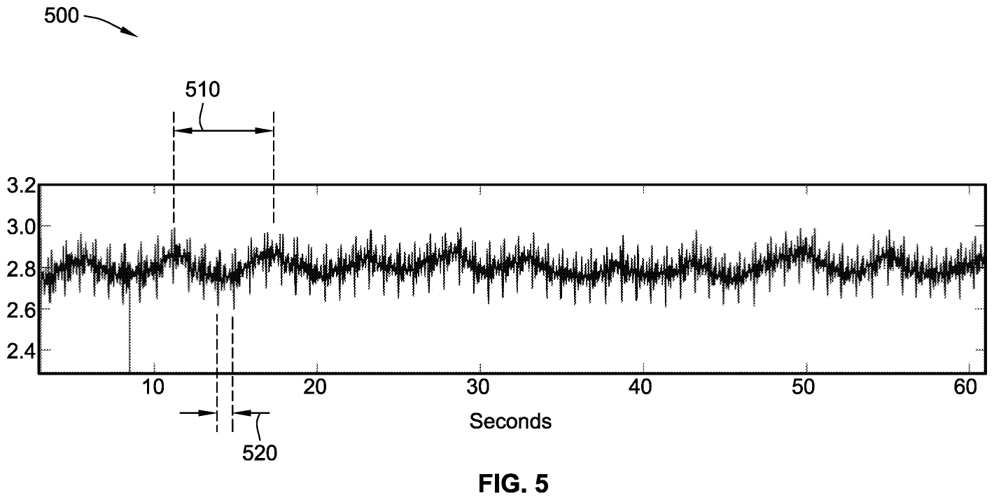

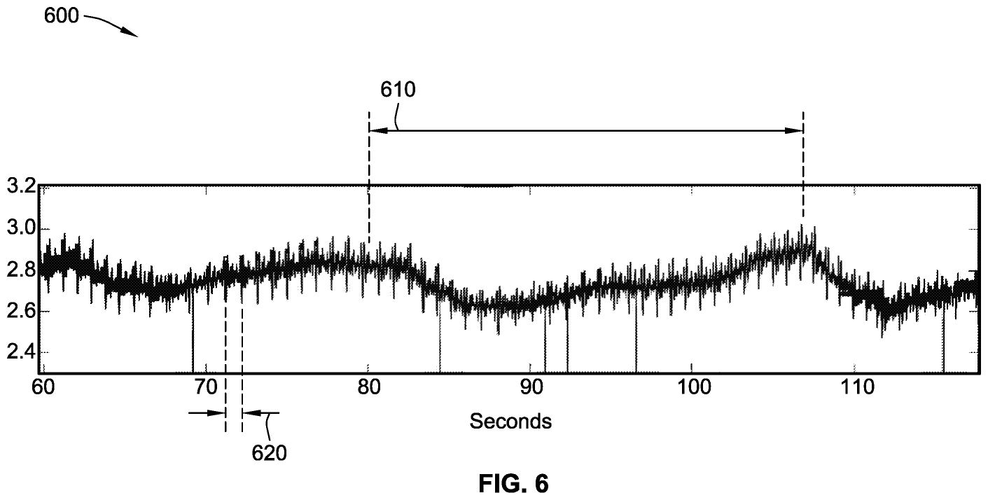

The first physiological data received during the first time period (step 210 of FIG. 2) is illustrated in FIG. 5, and the second physiological data received during the second time period (step 230 of FIG. 2) is illustrated in FIG. 6. The first physiological data (FIG. 5) and the second physiological data (FIG. 6) are generated using an accelerometer of a mobile device, such as the mobile device 170 of the system 100 (FIG. 1). The accelerometer is configured to detect the user's breathing and/or heart rate, which can be plotted as shown in FIGS. 5-6. In this example, the respiratory period 510 during the first time period is shorter than the respiratory period 610 during the second time period, because the user breathes slower during the second time period than the first time period. In this example of FIGS. 5-6, the cardiac period 520 and the cardiac period 620 are about the same. While in this example the first physiological data and the second physiological data are generated using an accelerometer, the first physiological data and/or the second physiological data may be generated by any sensor, such as one or more of the sensors 130 described herein. The corresponding data generated by such one or more of the sensors 130 may be analyzed to determine the percentage likelihood that the user has an untreated sleep disorder, whether the user has an untreated sleep disorder, and/or diagnose the user for the untreated sleep disorder. In some implementations, face scanning technology may be implemented to predict and/or diagnose positional OSA and/or positional snore.

Thus, in some implementations, the second respiration rate (determined at step 240 of FIG. 2) is less than the first respiration rate (determined at step 220 of FIG. 2). For example, the second respiration rate is at least 10% less, at least 20% less, at least 30% less, at least 40% less, at least 50% less, at least 60% less, or at least 70% less than the first respiration rate. In some implementations, having six breaths or fewer per minute is considered slow breathing. In some implementations, a target respiratory rate will be set for the second respiratory rate, such as six breaths per minute. Additionally, a tolerance may be set to assess if the target has been met, for example, if respiratory rate monitoring indicates that the user's respiratory rate stayed within a percentage of the target value for a predetermined time, then the test could be deemed acceptable, such that the user may be further guided or coached to achieve an acceptable test up until the point at which an acceptable test has been completed.

Referring back to FIG. 2, at step 260, the percentage likelihood that the user has an untreated sleep disorder (e.g., obstructive sleep apnea) is determined based at least in part on the first heart rate variability parameters determined at step 220 and the second heart rate variability parameters determined at step 240, such as the examples as follows.

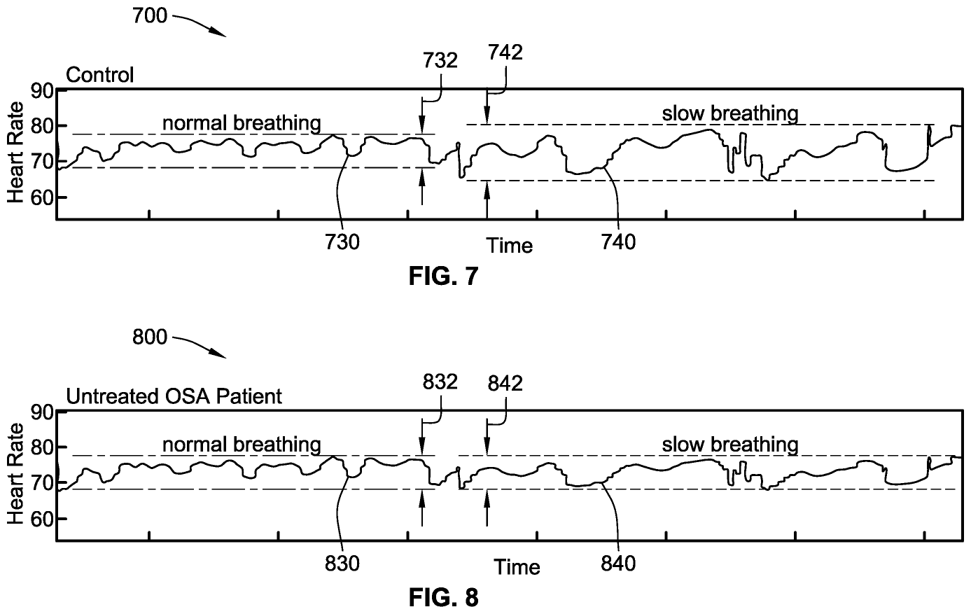

FIG. 7 illustrates physiological data associated with a user without a sleep disorder. The plot 700 shows the heart rates of the user without a sleep disorder during the first period of normal breathing, and during the second period of slow breathing. The heart rates 730 during the first time period has a minimum of about 68 beats per minute, and a maximum of about 78 beats per minute. Thus, the heart rate range 732 during the first time period is about 10 beats per minute. The heart rates 740 during the second time period has a minimum of about 66 beats per minute, and a maximum of about 80 beats per minute. Thus, the heart rate range 742 during the second time period is about 14 beats per minute.

FIG. 8 illustrates physiological data associated with a user having untreated OSA. The plot 800 shows the heart rates of the user having untreated OSA during the first period of normal breathing, and during the second period of slow breathing. The heart rates 830 during the first time period has a minimum of about 68 beats per minute, and a maximum of about 78 beats per minute. Thus, the heart rate range 832 during the first time period is about 10 beats per minute. The heart rates 840 during the second time period has a minimum of about 68 beats per minute, and a maximum of about 78 beats per minute. Thus, the heart rate range 742 during the second time period is about 10 beats per minute.

Thus, as illustrated in FIGS. 7-8, when breathing slows down, the heart rate range increases in a user without a sleep disorder. For a user having an untreated sleep disorder such as sleep apnea, the heart rate range does not increase to the same extent as that does in the user without a sleep disorder. In other words, when breathing slows down, the heart rate varies more in a user without a sleep disorder than that does in a user with an untreated sleep disorder.

Referring back to FIG. 2, in some implementations, step 210 and/or step 220 may be omitted, and only the second physiological data received during the second time period (step 230) is analyzed. During the second time period, the user's breathing is slowed (e.g., about or fewer than 6 breaths per minute). In some such implementations, at step 260, the percentage likelihood that the user has an untreated sleep disorder is determined based on the analyzed second physiological data. For example, in some implementations, if the heart rate range during the second time period does not exceed a threshold value (6 bpm, 7 bpm, 8 bpm, 9 bpm, 10 bpm, 11 bpm, 12 bpm, 13 bpm, 14 bpm, 15 bpm, 16 bpm, 17 bpm, 18 bpm, 19 bpm, 20 bpm, 21 bpm, 22 bpm), the user is determined to likely have untreated sleep apnea. In some implementations, the threshold value is adjusted based on the user's demographics. For example, respiratory coupling tends to be greater in young healthy people.

In some implementations, at step 250, the first heart rate variability parameters determined at step 220 are compared with the second heart rate variability parameters determined at step 240. For example, in some implementations, the heart rate range during the first time period (e.g., normal breathing) may be compared with the heart rate range during the second time period (e.g., slow breathing), such as the examples shown in FIGS. 7-8. The difference between the heart rate ranges, when exceeding a threshold, can indicate that the user does not have an untreated sleep disorder (e.g., the user is properly treated, or does not have a sleep disorder).

In some implementations, in response to the heart rate range for the second time period being no greater than the heart rate range for the first time period, at step 260, the percentage likelihood that the user has an untreated sleep disorder is determined to be greater than 40%, 50%, 60%, 70%, 80%, or 90%. Additionally or alternatively, at step 260, the user is determined to likely have an untreated sleep disorder. In some implementations, the determination that the user is likely to have an untreated sleep disorder is made in combination with other data, such as neck circumference, survey result, BMI, resting heart rate, etc., to produce a more accurate estimate of the presence and/or the type of sleep disorder.

In some implementations, at step 270, the first time period (step 210) may be labeled with a first time stamp, and the second time period (step 230) may be labeled with a second time stamp. In some implementations, the closer it is to the user wakes up from sleep (within 30 minutes, an hour, two hours, or three hours of the user waking up), the more accurate the determination is at step 260. For example, if the user has untreated mild OSA, the user may experience little increase in heart rate range in slow breathing shortly after waking up, but recover somewhat to experience closer to normal increase in heart rate range in slow breathing later in the day. If the user has untreated moderate to severe OSA, the user may continue to experience little increase in heart rate range in slow breathing even later in the day. In some implementations, the heart rate of the user is monitored for some time after the end of the deep breathing, and the slope and/or shape of the heart rate plot is analyzed for further determination.

To account for the difference in parasympathetic activity, in some implementations, the determination at step 260 may be adjusted according to the first time stamp and the time stamp. In some implementations, the determination at step 260 may be adjusted based on how well the user slept the night before. Additionally or alternatively, in some implementations, at step 280, a severity of the untreated sleep disorder may be determined based at least in part on (i) the first time stamp and the second time stamp labeled at step 270 and/or (ii) the percentage likelihood that the user has an untreated sleep disorder at step 260.

Additionally or alternatively, in some implementations, a plurality of measurements may be taken during the day to observe the changes in heart rate variability parameters for the user. One or more steps of 210-260 may be repeated throughout the day to determine the severity of the untreated sleep disorder.

Additionally or alternatively, in some implementations, a plurality of measurements may be taken over a longer period of time (for example, over multiple days, weeks, or months), to observe the changes in heart rate variability parameters for the user; and the history of results may be compared with other historical data, such as therapeutic data and/or lifestyle data, in order to establish patterns between therapeutic methods, lifestyle parameters, and/or sympathetic activity. In some implementations, established patterns may be used to guide the user toward therapies or behaviors that may reduce sympathetic activity, and reduce the risk of developing particular diseases. For example, the user or a care provider may be guided to consider a particular therapy for sleep disordered breathing, such as a positional sleep apnea therapy, or CPAP, based on the user's historical response to a range of therapies. In some implementations, changes in heart rate variability may be used as a metric to evaluate the effectiveness of a particular therapy, for example, if after trialing a particular therapy for sleep apnea, the heart rate variability does not increase, the user or clinician may be guided to investigate the effectiveness of alternative therapies, or alternative therapy settings or modes.

Additionally or alternatively, at step 290, additional physiological data associated with the user during an additional time period is received. At step 292, the additional physiological data received at step 290 is analyzed to determine (i) an additional respiration rate for the additional time period, (ii) an additional plurality of sample heart rate values, and (iii) additional heart rate variability parameters for the additional time period. The additional respiration rate (step 290) is less than the first respiration rate (step 220). In some such implementations, the severity of the untreated sleep disorder at step 280 may be determined based at least in part on the first heart rate variability parameters (step 220), the second heart rate variability parameters (step 240), and the additional heart rate variability parameters (step 292).

In some implementations, (i) the first physiological data (step 210), (ii) the second physiological data (step 230), and/or (iii) the additional physiological data (step 290) is received from a mobile device coupled to the user's chest (FIG. 4), a heart rate sensor, a pulse sensor (e.g., a pulse oximeter, an ECG device), or any combination thereof. In some such implementations, any of these physiological data generated by one or more of these sensors, and/or one or more of the other sensors described herein may be analyzed to determine the percentage likelihood that the user has an untreated sleep disorder, whether the user has an untreated sleep disorder, and/or diagnose the user for the untreated sleep disorder.

In some implementations, at step 262, an indication of the percentage likelihood that the user has an untreated sleep disorder determined at step 260 is displayed (e.g., on a display device such as the display device 172 of the system 100). For example, in some implementations, in response to the determined percentage likelihood that the user has an untreated sleep disorder exceeding 50%, the indication displayed at step 262 includes that the user is likely to have an untreated sleep disorder.

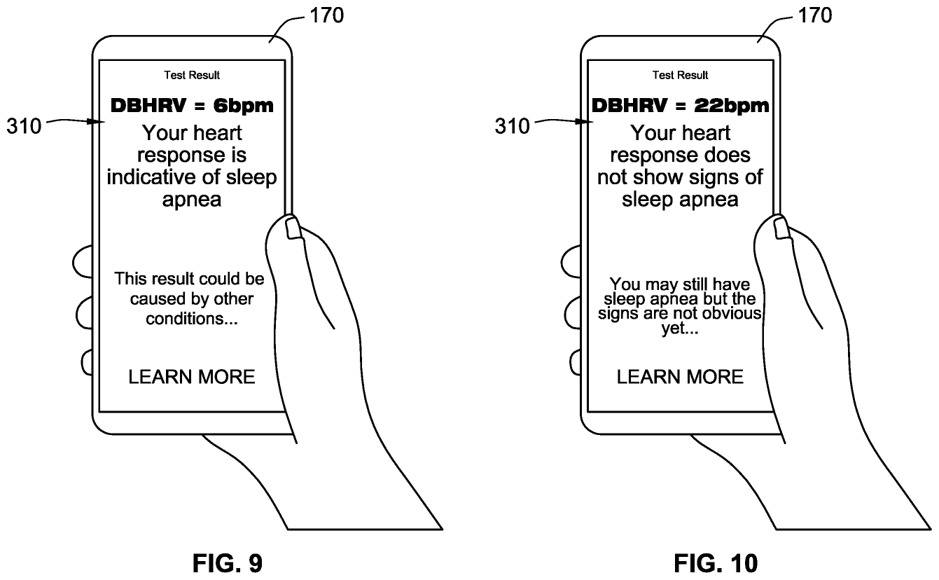

FIG. 9 illustrates a displayed indication to a user who is likely to have untreated OSA. The user in FIG. 9 may experience low deep breathing heart rate variability (e.g., the heart rate range during slow breathing does not exceed the threshold increase over the heart rate range during normal breathing). An indication 310 may be displayed on the mobile device 170, showing a deep breathing heart rate variability (DBHRV) as 6 beats per minute. For that user, the heart response is indicative of sleep apnea. The indication 310 may further include information such as “this result could be caused by other conditions” to prompt the user to learn more about other possible causes.

FIG. 10 illustrates a displayed indication to a user who is unlikely to have untreated OSA. The user in FIG. 10 may experience normal deep breathing heart rate variability (e.g., the heart rate range during slow breathing exceeds the threshold increase over the heart rate range during normal breathing). An indication 310 may be displayed on the mobile device 170, showing a deep breathing heart rate variability (DBHRV) as 22 beats per minute. For that user, the heart response does not show signs of sleep apnea. The indication 310 may further include information such as “you may still have sleep apnea but the signs were not obvious during this test” to increase awareness for the user to learn more about sleep disorders.

In some implementations, positional data associated with the user is also received. The received positional data may be analyzed to determine a body position of the user. Based at least in part on the determined body position of the user and the determined percentage likelihood that the user has an untreated sleep disorder (step 260), the user is caused to change body position. In some implementations, a sound or a vibration may be communicated to the user. For example, in some such implementations, the level of the sound or the vibration communicated to the user may be proportional to the determined severity of the untreated sleep disorder (step 280). Additionally, or alternatively, in some implementations, the level of the sound or the vibration communicated to the user may gradually increase to awaken the user. For some users, how difficult it is to arouse from sleep is associated with and/or correlated to how severe the untreated sleep disorder is.

In an example, the system may gradually increase the level of the stimulus, and monitor the response of the user via any of the sensors monitoring any aspect of the user's condition (e.g., an accelerometer might monitor the user's movement). In some implementations, historical data correlating the stimulus level to the user's response may be used to develop a model of the user's arousability. Further, the derived arousability may be used to evaluate the efficacy of a treatment. For example, in many cases, users with untreated sleep disorders may be relatively more difficult to arouse from sleep, due to deprivation of good quality sleep. As a result of effective treatment, the users may gradually become more easily aroused.

In some implementations, the frequency of the sound or vibration being transmitted may ramp up if it is detected that the user has not changed body position. In some implementations, the frequency of the sound or vibration being transmitted is adjusted proportionally to the sleep stage of the user. For example, if lightly sleeping, the stimulus may wake the user up. In some implementations, the prompt for the user to change body position requires one or both of the following conditions to be met: (i) the user is supine, and (ii) one or more respiratory events such as snoring, flow limitation, hypopnea, and apnea are detected.

In some implementations, one or more methods disclosed herein may be incorporated as part of a low-cost application (such as a low-cost positional OSA application), or part of a version of an OSA app. In some such implementations, the application is configured to perform a test, using one or more steps from the methods disclosed herein, to determine if the current therapy or the current therapy mode is suitable, before upgrading to a version designed more for long-term use. In some implementations, one or more steps from the methods disclosed herein may be incorporated as part of a snore therapy application. For example, the application is configured to detect snore sounds, using one or more sensors 130 disclosed herein (such as a microphone on the mobile device 170). In some such implementations, the mobile device 170 only alerts the user (e.g., by vibrating or playing a sound) when snore is detected. Alternatively, in some such implementations, the mobile device 170 only alerts the user when snore is detected and the patient is at one or more specific body positions (e.g., when the patient is in the supine body position, where snoring is indicative of an obstructed airway or a worse obstructed airway).

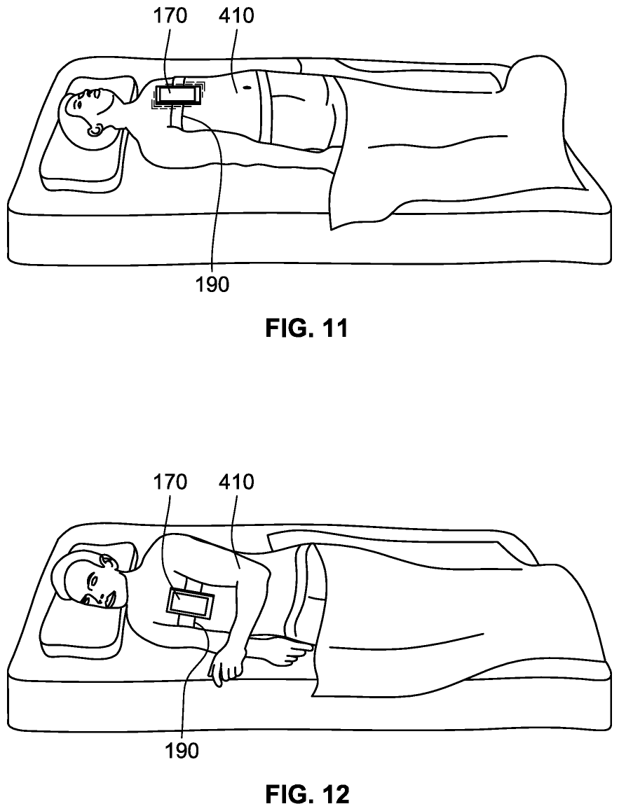

As an example, FIG. 11 shows the user 410 wearing the mobile device 170 and in a supine body position. The mobile device 170 may be coupled to the user 410 using the strap 184. The user may experience positional sleep apnea in the supine position. Thus, the mobile device 170 may buzz the user, e.g. by playing a sound or vibrating, to cause to user to turn to their side, as shown in FIG. 12.

In some implementations, based at least in part on the determined percentage likelihood that the user has an untreated sleep disorder, the user may be instructed to wear a treatment device, such as a respiratory therapy system. The treatment device may be configured to generate sensor data, and/or cause a sound or a vibration to be communicated to the user. In some implementations, the sensor data may include positional data associated with the user. Additionally or alternatively, the sensor data is generated using an acoustic sensor (e.g., a microphone), and/or a motion sensor (e.g., an accelerometer, a gyroscope, a magnetometer, or any combination thereof). The generated sensor data may then be analyzed to determine a sleep disorder event associated with the user, chest movement of the user, a heart rate of the user, or any combination thereof.

People typically change sleep positions regularly throughout a sleep session, and usually adopt one of a number of different positions for periods during a segment of sleep time. Whether it's sleeping completely flat (e.g., in a horizontal position), reclined, or sitting upright; or whether it's lying on their stomach (e.g., in a prone position), on their back (in a supine position), or on the left or right side.

Breathing conditions for an individual's body are different when the individual is lying down as compared to when the individual is standing up. When the individual is sitting or is on the feet, the individual's airway is pointing downward, leaving breathing and airflow relatively unrestricted. However, when the individual settles down to sleep, the individual's body is imposed to breathing in a substantially horizontal position, meaning that gravity is now working against the airway. Sleep apnea and snoring can occur when the muscular tissues in the top airway relax and the individual's lungs get limited air to breathe via the nose or throat. While the process of breathing is the same at night, the individual's surrounding tissues can vibrate, causing the individual to snore. Even relaxed muscles can cause sleep apnea because the total blockage of the airway hampers breathing fully, forcing the individual to wake up in the middle of sleep. As a result, it is important for the individual to sleep in a position that best supports the individual's breathing patterns. For example, some individual may benefit from sleeping in a reclined position rather than completely horizontal relative to ground.

Sleeping in the supine position can often be problematic for those who have snoring problems, breathing problems, or sleep apnea. This happens because the gravitational force enhances the capacity of the jaw, the tongue, and soft palate to drop back toward the throat. It narrows the airways and can cause troubles while breathing.

Sleeping in the prone position may seem like an alternative to the gravity issue as the downward force pulls the tongue and palate forward. While this is true to an extent, when sleeping in this position, the individual's nose and mouth can become blocked by the pillow. It may affect the individual's breathing. Apart from this, it may also cause neck pain, cervical problems, or digestion problems, which in turn affect the individual's sleep quality.

Some studies suggest that sleeping on the side may be the most ideal position for snoring and sleep apnea sufferers. Because when the individual's body is positioned on its side during rest, the airways are more stable and less likely to collapse or restrict air. In this position, the individual's body, head and torso are positioned on one side (left or right), arms are under the body or a bit forward or extended, and legs are packed with one under the other or slightly staggered. While both lateral (left and right) sides are considered as good sleeping positions, for some the left lateral position may not be an ideal one. That's because while sleeping on the left side, the internal organs of the body in the thorax can face some movement. And the lungs may add more weight or pressure on the heart. This can affect the heart's function, and it can retaliate by activating the kidneys, causing an increased need for urination at night. The right side, however, puts less pressure on the vital organs, such as lungs and heart. Sleeping on a particular side can also be ideal if a joint (often shoulder or hip) on the individual's other side is causing pain.

When an individual has sleep apnea or other breathing disorders, getting a good and peaceful sleep becomes difficult. However, choosing the right sleeping position can help the user get comfortable and at the same time help overcome the breathing problems that the individual usually face while sleeping. Thus, according to some implementations of the present disclosure, systems and methods are provided to cause the user to change body position if they are sleeping in an undesired body position (e.g., supine). Positional therapy not only can provide treatment for users with mild OSA, but also for users already undergoing another therapy who could have a more comfortable option.

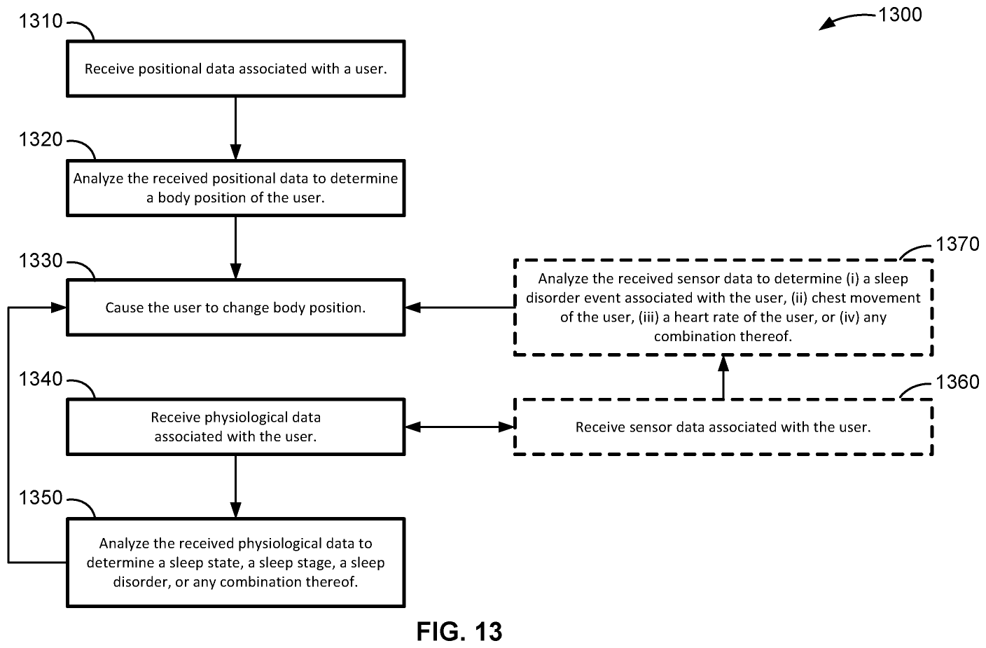

Referring to FIG. 13, a flow diagram for a method 1300 for monitoring a body position of a user is disclosed. At step 1310, positional data associated with a user is received. In some implementations, the positional data associated with the user is received from a mobile device (e.g., a mobile device 170 of the system 100) coupled to the user's chest, a heart rate sensor, a pulse sensor, or any combination thereof.

At step 1320, the positional data received at step 1310 is analyzed to determine a body position of the user. In some implementations, the body position is generally supine, generally left lateral, generally right lateral, or generally prone. At step 1330, based at least in part on the body position of the user determined at step 1320, the user is caused to change body position. In some implementations, the causing thes user to change the body position includes causing a sound or a vibration to be communicated to the user. As an example, FIG. 11 shows the user 410 wearing the mobile device 170 and in a supine body position. The mobile device 170 may be coupled to the user 410 using the strap 184. The user may experience positional sleep apnea in the supine position. Thus, the mobile device 170 may buzz the user to cause to user to turn to their side, as shown in FIG. 12.

In some implementations, at step 1340, physiological data associated with the user is received. At step 1350, the physiological data received at step 1340 is analyzed to determine a sleep state, a sleep stage, a sleep disorder, or any combination thereof. For example, in some implementations, the sleep state is awake or asleep. Additionally or alternatively, in some implementations, the sleep state is fully awake, relaxed awake, drowsy, dozing off, asleep in light sleep, asleep in deep sleep, or asleep in rapid eye movement. In some implementations, the sleep stage is stage N1, stage N2, stage N3, slow wave, or rapid eye movement (REM). In some implementations, the sleep disorder includes periodic limb movement disorder, obstructive sleep apnea, central sleep apnea, positional sleep apnea, or any combination thereof. In some implementations, the causing the user to change body position at step 1330 is further based at least in part on the sleep state, the sleep stage, and/or the sleep disorder determined at step 1350.

In some implementations, at step 1360, sensor data associated with the user is received from a mobile device. In some such implementations, the sensor data at step 1360 is the same as, or similar to, the physiological data at step 1340. In some other implementations, the sensor data at step 1360 is separate and distinct from the physiological data at step 1340. At step 1370, the sensor data received at step 1360 is analyzed to determine (i) a sleep disorder event associated with the user, (ii) chest movement of the user, (iii) a heart rate of the user, or (iv) any combination thereof, which may then be used to monitor the user and/or determine when to cause the user to change body position at step 1330.

In some implementations, upon corrective training for the body position using the method 200 and/or the method 1300, the user may be prompted to check their heart rate parameters (method 200) at a predetermined later date to verify if the positional treatment has resulted in a reduction in the heart rate range (and therefore a reduction in likelihood of having OSA). If not, the user may be prompted to go on sleep therapy. Additionally or alternatively, in some implementations, the cardio-respiratory signal (e.g., received from the accelerometer) may be used as input to the therapy algorithm. For example, signs of sleep apnea (e.g., increased respiratory effort, increased heart rate, apnea, hypopnea, snore sounds), as well as body position before activating therapy, may be monitored. Such monitoring can lead to less unnecessary interruptions during the night. In some implementations, the angle of the phone may provide an indication of respiratory effort or resistance (e.g. with paradoxical breathing, which can happen with high airway re si stance).

In some implementations, the accelerometer signals (or similar) that indicate excessive movement and/or noise that masks both respiratory and cardiogenic chest movement is indicative of wakeful movement. During periods of the user being relative still, the cardio respiratory signals contain features indicative of the user's sleep state. For example, in some implementations, regularity of breathing, rate of breathing, depth of breathing, heart rate, heart rate variability, and/or reduction of magnitude of chest movement, at respiratory rate for brief periods (e.g., 10-60 seconds) is indicative of apnea or hypopnea. One or more of these features can be collected over various time frames and/or used to train a system (e.g., support vector machine, neutral network, etc.) to identify the sleep state and/or classify the sleep state.

In some implementations, such as for positional therapy, the therapy can be disabled and/or paused with the detection of a particular sleep state (e.g., awake), and resumed with the detection of another state (e.g., sleep onset). In some such implementations, the transition from awake to sleep onset can be characterized by (i) an increase in respiratory rate, (ii) a reduction in respiratory amplitude, (iii) the establishment of a more regular respiratory rate, or (iv) any combination thereof.

In some implementations, multiple therapy modes can be combined. For example, a positional therapy can be combined with a positive airway pressure therapy, such that the pressure requirements of the positive airway pressure therapy may be reduced in certain body positions. In some implementations, a position monitoring application can be combined with a positive airway therapy, such that the user position is factored into an algorithm for determining the target therapy pressure. For example, the target therapy may be increased when the user transitions to a horizontal position, or the target pressure may be increased when the user transitions from a prone or side position (or any other position) to a supine position. Similarly, the target pressure may be reduced when the user transitions away from a supine position. In some implementations, demographic data, and/or historical therapy data may be used to estimate the magnitude in change in target pressure to be applied at a particular transition in position.

Studies have suggested that positional OSA patients, compared to non-positional OSA patients, have a more backward positioning of the lower jaw, lower facial height, longer posterior airway space measurements, and a smaller volume of lateral pharyngeal wall tissue. Such characteristics of the positional OSA patients result in a greater lateral diameter and elliptoid shape of the upper airway. In addition, positional OSA patients tend to have a smaller neck circumference. Thus, it is suggested that even though the anterior-posterior diameter in both positional OSA patients and non-positional OSA patients is reduced as a result of the effect of gravity in the supine position, there is sufficient preservation of airway space and avoidance of complete upper airway collapse because of the greater lateral diameter in positional OSA patients. Thus, it is advantageous to predict and/or diagnose patients with positional OSA, and generate treatment plans and/or adjust treatment parameters accordingly. In some implementations, the body position of the user is taken into account when making such treatment plans and/or adjusting such treatment parameters. In some implementations, one or more steps of the methods disclosed herein may be incorporated into an application that integrates prediction, screening, diagnosis, and therapy altogether.

In some implementations, one or more steps of the methods disclosed herein may be incorporated into distributed systems for snore and/or positional OSA prediction, screening, diagnosis, and/or treatment. In one example, a first user device, such as a smartwatch may pick up a heart rate of the user, or any other physiological parameters as disclosed herein. A separate sensor (such as an accelerometer) wireless from the first user device on the chest and/or the head of the user is activated to determine a torso and/or head position. An analysis is then performed to determine if the head position or the torso position or both are important for the user. In some such implementations, the user device may also be configured to buzz as needed to alert the user.

Generally, the methods 200 and 1300 can be implemented using a system having a control system with one or more processors, and a memory storing machine readable instructions. The controls system can be coupled to the memory; the methods 200 and 1300 can be implemented when the machine readable instructions are executed by at least one of the processors of the control system. The methods 200 and 1300 can also be implemented using a computer program product (such as a non-transitory computer readable medium) comprising instructions that when executed by a computer, cause the computer to carry out the steps of the methods 200 and 1300.

While the system 100 and the methods 200 and 1300 have been described herein with reference to a single user, more generally, the system 100 and the methods 200 and 1300 can be used with a plurality of users simultaneously (e.g., two users, five users, 10 users, 20 users, etc.). For example, the system 100 and methods 200 and 1300 can be used in a cloud monitoring setting.

While some examples of the system 100 and the methods 200 and 1300 have been described herein with reference to determining one or more untreated sleep disorders, more generally, the system 100 and the methods 200 and 1300 can be used to determine one or more health-related issues, such as any disease or condition that increases sympathetic activity, examples of which include COPD, CVD, somatic syndromes, etc.

Referring again to FIG. 14, a portion of the system 100 (FIG. 1), according to some implementations, is illustrated. The user 410 of the respiratory therapy system 120 and a bed partner 1420 are located on a bed 1430 and laying on a mattress 1432. In some implementations, the user 410 may rest the head on a pillow 1434. The user interface 124 (also referred to herein as a mask, e.g., a full face mask) can be worn by the user 410 during a sleep session. The user interface 124 is fluidly coupled and/or connected to the respiratory therapy device 122 via the conduit 126. In turn, the respiratory therapy device 122 delivers pressurized air to the user 410 via the conduit 126 and the user interface 124 to increase the air pressure in the throat of the user 410 to aid in preventing the airway from closing and/or narrowing during sleep. The respiratory therapy device 122 can be positioned on a nightstand 1440 that is directly adjacent to the bed 1430 as shown in FIG. 14, or more generally, on any surface or structure that is generally adjacent to the bed 1430 and/or the user 410.

The blood pressure device 180 is generally used to aid in generating cardiovascular data for determining one or more blood pressure measurements associated with the user 410. The blood pressure device 180 can include at least one of the one or more sensors 130 to measure, for example, a systolic blood pressure component and/or a diastolic blood pressure component.

The activity tracker 190 is generally used to aid in generating physiological data for determining an activity measurement associated with the user 410. The activity tracker 190 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 190 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 190 is coupled (e.g., electronically or physically) to the user device 170.

In some implementations, the activity tracker 190 is a wearable device that can be worn by the user 410, such as a smartwatch, a wristband, a ring, or a patch. For example, referring to FIG. 14, the activity tracker 190 is worn on a wrist of the user 410. The activity tracker 190 can also be coupled to or integrated a garment or clothing that is worn by the user 410. Alternatively, still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory device 114, the respiratory therapy system 120, the user device 170, and/or the blood pressure device 180.

Everyone has their own preferences for sleeping. Whether it's sleeping completely flat (e.g., in a horizontal position), reclined, or sitting upright; or whether it's lying on their stomach (e.g., in a prone position), on their back (in a supine position), or on your left or right side.

Breathing conditions for an individual's body are different when the individual is lying down as compared to when the individual is standing up. When the individual is sitting or is on the feet, the individual's airway is pointing downward, leaving breathing and airflow relatively unrestricted. However, when the individual settles down to sleep, the individual's body is imposed to breathing in a substantially horizontal position, meaning that gravity is now working against the airway. Sleep apnea and snoring can occur when the muscular tissues in the upper airway (or other muscles such as the soft palate, tongue, etc.) relax and the individual's lungs get limited air to breathe via the nose or throat. While the process of breathing is the same at night, the individual's surrounding tissues can vibrate, causing the individual to snore. Sometimes relaxed muscles can cause sleep apnea because some blockage of the airway hampers breathing fully, forcing the individual to wake up in the middle of sleep. As a result, it is important for the individual to sleep in a position that best supports the individual's breathing patterns. For example, some individual may benefit from sleeping in a reclined position rather than completely horizontal relative to ground.

Sleeping in the supine position can often be problematic for those who have snoring problems, breathing problems, or sleep apnea. This happens because the gravitational force enhances the capacity of the jaw, the tongue, and soft palate to drop back toward the throat. This may narrow or collapse the airways and can cause troubles while breathing.

Sleeping in the prone position may seem like an alternative to the gravity issue as the downward force pulls the tongue and palate forward. While this is true to an extent, when sleeping in this position, the individual's nose and mouth can become blocked by the pillow. It may affect the individual's breathing. Apart from this, it may also cause neck pain, cervical problems, or digestion problems, which in turn affect the individual's sleep quality.

Some studies suggest that sleeping on the side may be the most ideal position for snoring and sleep apnea sufferers. Because when the individual's body is positioned on its side during rest, the airways are more stable and less likely to collapse or restrict airflow. In this position, the individual's body, head and torso are positioned on one side (left or right), arms are under the body or a bit forward or extended, and legs are packed with one under the other or slightly staggered. While both lateral (left and right) sides are considered as good sleeping positions, for some the left lateral position may not be an ideal one. That's because while sleeping on the left side, the internal organs of the body in the thorax can face some movement. And the lungs may add more weight or pressure on the heart. This can affect the heart's function, and it can retaliate by activating the kidneys, causing an increased need for urination at night. The right side, however, puts less pressure on the vital organs, such as lungs and heart. Sleeping on a particular side can also be ideal if a joint (often shoulder or hip) on the individual's other side is causing pain.

When an individual has sleep apnea or other breathing disorders, getting a good and peaceful sleep becomes difficult. However, choosing the right sleeping position can help the user get comfortable and at the same time help overcome or alleviate the breathing problems that the individual usually face while sleeping. Thus, according to some implementations of the present disclosure, systems and methods are provided to cause the user to change body position if they are sleeping in an undesired body or head position (e.g., supine). Positional therapy not only can provide treatment for users with mild OSA, but also for users already undergoing another therapy who could have a more comfortable and efficacious option.

Still referring in FIG. 1, in some implementations, the system 100 further includes a communications module 182, a strap 184, and a passive treatment device 192. In some implementations, the treatment device 192 is a smartwatch. In some implementations, the treatment device 192 is communicatively coupled to an electronic device (e.g., via the communications module 182), and is configured to transmit data associated with the user to the electronic device. For example, in some such implementations, the electronic device is a mobile phone. In some other implementations, the electronic device is a respiratory therapy device (e.g., the respiratory therapy device 122) configured to supply pressurized air to an airway of the user. The data transmitted from the treatment device 192 is then configured to cause a setting of the respiratory therapy device 122 to be adjusted. For example, the setting may be a pressure setting of the respiratory therapy device 122.

According to some implementations of the present disclosure, a wearable device may include the treatment device 192 and the strap 184 coupled to the treatment device 192. For example, in some such implementations, the strap 184 may be at least a portion of a headband, an eye mask, a face mask, a pair of headphones, or the like. As shown in FIG. 14, the user 410 wears the treatment device 192 (FIG. 1) that is secured to his head via the strap 184.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and optionally the user device 170. As a further example, a fourth alternative system includes the strap 184, the passive treatment device 192, and at least one of the one or more sensors 130 and does not include the respiratory therapy system 120. As yet a further example, a fifth alternative system includes the control system 110, the memory device 114, the respiratory therapy system 120, at least one of the one or more sensors 130, and the passive treatment device 192. Thus, various systems can be formed using any portion or portions of the components shown and described herein and/or in combination with one or more other components.

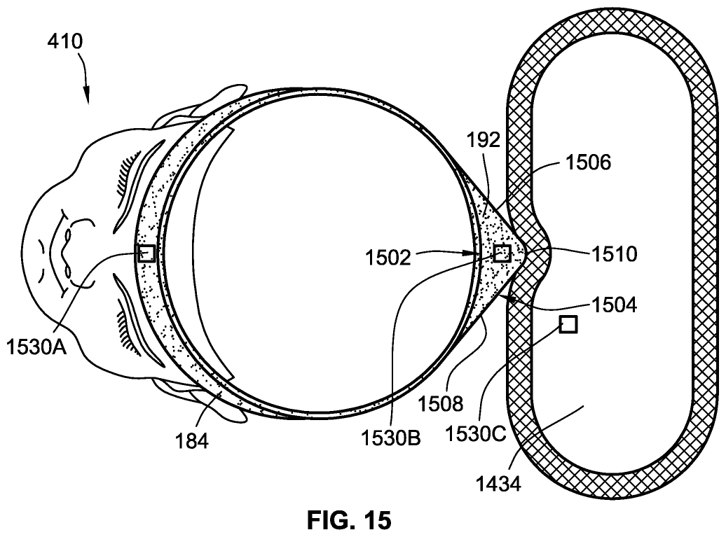

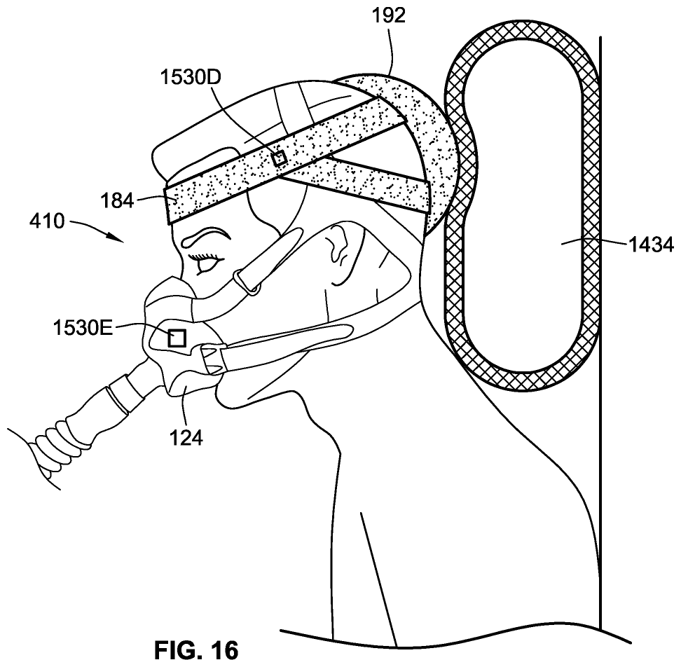

Referring generally to FIGS. 15-16, a top perspective view of the user 410 wearing the treatment device 192 is shown in FIG. 15, whereas a side view of the user 410 wearing the treatment device 192 is shown in FIG. 16, according to some implementations of the present disclosure. The strap 184 is configured to be worn around the head of the user 410 to secure the treatment device 192 to the back of the head of the user 410. In some implementations, the treatment device 192 is removably coupled to the strap 184. For example, in some such implementations, the treatment device 192 is configured to snap onto the strap 184. In some other implementations, the treatment device 192 is permanently coupled to the strap 184. In some implementations, the treatment device 192 may be worn without a respiratory therapy device, such as shown in FIG. 15. In some implementations, the user 410 may wear the treatment device 192 along with a respiratory therapy device, such as shown in FIG. 16.

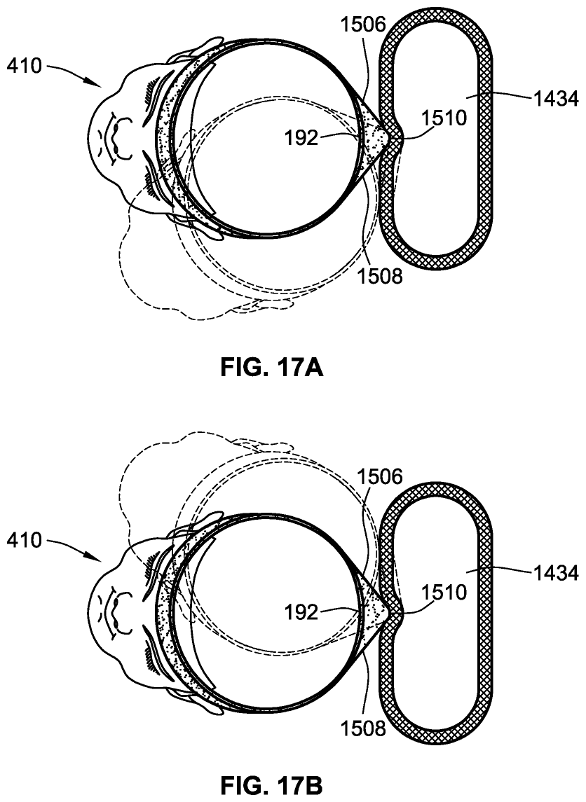

As shown in FIG. 15, in some implementations, the treatment device 192 is generally semi-ellipsoidal. For example, in some such implementations, at least a portion of the treatment device 192 is shaped as a cone. The treatment device 192 includes a concave surface 1502 and a convex surface 1504. The concave surface 1502 is configured to contact the back of the head of the user 410. The head of the user 410 is facing upright when the treatment device 192 is positioned about the vertex 1510 of the convex surface 1504. The head of the user 410 is facing toward either side (FIGS. 17A-17B) when the treatment device 192 is positioned on the either side (1506 or 308) of the convex surface 1504.

To aid in passively urging the user to sleep on his side, the treatment device 192 is bi-stable on the convex surface 1504, such that the treatment device 192 is stable when positioned on either side (1506 or 308) of the convex surface 1504, and unstable when positioned about a vertex 1510 of the convex surface 1504. In some implementations, a thickness of the treatment device 192 measured from a center of the concave surface 1502 to the vertex 1510 of the convex surface 1504 is between 2 cm to 8 cm. In some such implementations, the thickness of the treatment device 192 measured from the center of the concave surface 1502 to the vertex of the convex surface is about cm.

Additionally or alternatively, in some implementations, the treatment device may be weighted and/or powered to aid the user 410 in moving away from facing upright. For example, a weighted wearable device may include a weighted treatment device and the strap 184. The weighted treatment device may include a concave surface and an opposite surface that is not necessarily convex. The concave surface is configured to contact a back of a head of a user, similar to the concave surface 1502. The weighted treatment device is bi-stable on the opposite surface due to its weight distribution, such that the weighted treatment device is stable when positioned on either side of the opposite surface, and unstable when positioned about a center of the opposite surface. The strap 184 is configured to be worn around the head of the user to secure the weighted treatment device to the back of the head of the user, in a similar manner as described with reference to the treatment device 192.

In some implementations, the wearable device further includes a sensor configured to measure and/or determine physiological data associated with the user 410. The physiological data is described above with reference to FIG. 1. The sensor maybe the same as, or similar to, one or more sensors 130 shown in FIG. 1. The sensor may be positioned at any suitable location. For example, in some implementations, the sensor is coupled to or integrated in the strap 184 at location 1530A. Additionally or alternatively, in some implementations, the sensor is coupled to or integrated in the treatment device 192 at location 1530B. Further additionally or alternatively, in some implementations, the sensor is coupled to or integrated in the pillow 1434 at location 1530C.

Referring to FIG. 16, in some implementations, the sensor is coupled to or integrated in the strap 184 at location 1530D. For example, in some such implementations, the sensor may be a pulse-oximeter (e.g., the same as, or similar to, the oxygen sensor 168 of the system 100) coupled to the strap 184 and configured to be in contact with a temple of the user 410. Additionally or alternatively, in some implementations, the sensor is coupled to or integrated in the user interface 124 at location 1530E, or in any other component of a respiratory therapy system.

In some implementations, the sensor is configured to measure and/or determine a movement of the user, a position or orientation of the user (e.g. supine, prone, on their side, upright), a pulse of the user, a pulse rate of the user, a pulse rate variability of the user, a pulse wave amplitude of the user, a pulse waveform of the user, a pulse oxygen saturation of the user, a respiratory rate of the user, a respiratory waveform of the user, an ECG, EEG, or EMG of the user, a measure of vascular dilation of the user, or any combination thereof. For example, in some such implementations, the sensor is an accelerometer (e.g., the same as, or similar to, the motion sensor 138 of the system 100).

In some implementations, the accelerometer is positioned in contact with or coupled to the skin of the head or face of the user, such as a surface of the head or face from which the orientation of the head can be derived, for example, the temple, forehead, or the side, back, or top of the head. In some implementations, the accelerometer may be positioned in contact with or coupled to the head of the user, such as the forehead (to detect the position of the head) or the mandible (to detect the position of the head and/or movement of the jaw). In other implementations, it may be preferable to have a sensor in contact with or coupled to a region of the head known to have a strong pulse, such as at the temple or along the carotid artery. In one example, the sensor may be configured to determine parameters related to the pulse of the user. In yet another example, it may be desirable to have a sensor located under the nose, or near the nose or mouth or anywhere along the user airways, and configured to determine parameters related to the user breathing. In some implementations, the accelerometer is a tri-axial accelerometer. In some implementations, the accelerometer is configured to generate positional data associated with the head of the user. In some implementations, the accelerometer is positioned in contact with or coupled to the skin of the head, neck, or face, and proximal to an artery of the head, neck, or face, such as any of the carotid, facial, auricular, occipital, or temporal arteries, and the accelerometer is configured to measure and/or determine a pulse of the user.

Additionally or alternatively, in some implementations, the sensor may consist of a single sensing element, or an array or distribution of sensing elements and be configured to measure and/or determine EEG and be positioned near one or more regions of the brain of interest such as the frontal, parietal, temporal, or occipital lobes, or the cerebellum; or to measure ECG and be positioned with at least one element coupled to the skin away from the sagittal plane; or to measure EMG, such as muscle activity related to respiration or jaw movement, and be placed in contact with the skin near the muscles of interest such as the muscles that control the jaw; or to measure EOG and be placed in contact with skin near the muscles that control eye movement, or any combination thereof. Further additionally or alternatively, in some implementations, the sensor is configured to measure and/or determine apnea, position, heart rate, heart-rate variability, or any combination thereof. For example, in some implementations one or more sensors can be positioned on the torso, in contact or coupled to the thorax, such that measurement of pressure, displacement, or their derivative signals (e.g., a signal might be derived from one or more other signals, including, but not limited to, a derivative in the sense of a gradient or rate of change signal, such as described in integral calculus) may be indicative of mechanical functions of the heart or cardiovascular system, as well as the position or orientation of the thorax relative to the local gravitational field. Similarly, a sensor in the form of an electrode may alternatively, or additionally measure electrical activity on the thorax associated with electrical activity of the heart or muscles such as the diaphragm. Similarly, and alternatively, or additionally, one or more sensors may be in contact with or coupled to the head, such that they can measure signals associated with the cardiovascular vessels of the head, or electrical activity generated at the heart, or electrical activity associated with muscles of the head, such as those that control respiratory patency, or jaw or eye movement. Additionally, measurements may be taken that are indicative of the position of the head. In yet further implementations, the position of the head and the thorax may both be measured, and compared with each other and/or other measurements, such as those related to respiratory air flow, or blood oxygen saturation. For example, it may be desirable to link any combination of head position and thorax position with improved or worsening severity of sleep apnea.

According to some implementations of the present disclosure, the disclosed devices may include, either in combination with other components described, or not, a means of transmitting an audible signal to the user, either via one or more bone conduction transducers, one or two in ear audio speakers, or one or two over the ear audio speakers. Further, the in-ear or over-ear speakers may be configured to form a low pressure sealing surface such that a sealed cavity is created between the speaker and the user. In some implementations, the sealed cavity may include an external auditory canal, or there may be two cavities each including a different auditory canal, and in other implementations a cavity may entirely enclose one of the ears. In another embodiment, there may be two separate cavities formed around and entirely enclosing each ear separately. In yet further implementations, the cavities may be fitted with one or more sensors, such as accelerometers, electrodes, pressure sensors, and temperature sensors, for the purpose of measuring biological parameters. For example, a pressure sensor coupled to the cavity may be configured to determine a blood pulse waveform, or any other parameters associated with the pulse, or a volume of air in the cavity, such that it is possible to infer changes in the local volume of blood and hence with different methods of filtering (such as low-pass or moving average filtering) determine pulse volume, or local vascular volume.

According to some implementations of the present disclosure, a method provides generating physiological data associated with the user via any of the treatment devices disclosed above. The method further provides determining whether the user has sleep apnea based at least in part on the generated physiological data associated with the user. In some implementations, the method further provides recommending a treatment option associated with the user.

Referring generally to FIGS. 17A-17B, as disclosed above, the treatment device 192 is bi-stable on the convex surface 1504, such that the treatment device 192 is stable when positioned on either side (1506 or 308) of the convex surface 1504, and unstable when positioned about the vertex 1510 of the convex surface 1504. FIG. 17A illustrates that the user 410 wearing the treatment device 192 moves from facing upright (solid lines) to facing left (dotted lines), according to some implementations of the present disclosure. As shown, it is uncomfortable for the user 410 to lay upright, and the user 410 is therefore urged to move to the left, where the side 308 of the convex surface 1504 rests along the pillow 1434. Similarly, FIG. 17B illustrates that the user 410 wearing the treatment device 192 moves from facing upright (solid lines) to facing right (dotted lines), according to some implementations of the present disclosure. As shown, it is uncomfortable for the user 410 to lay upright, and the user 410 is therefore urged to move to the right, where the side 1506 of the convex surface 1504 rests along the pillow 1434.

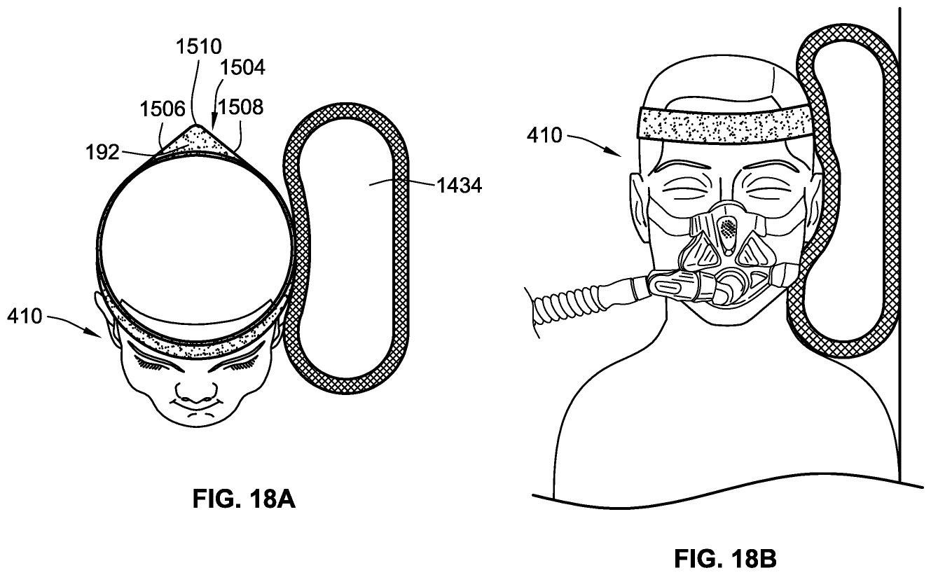

While the user 410 may be comfortable facing partially left (FIG. 17A) or partially right (FIG. 17B), the user 410 will also be comfortable sleeping completely on the left side or on the right side, while wearing the treatment device. For example, FIG. 18A is a top perspective view of the user 410 wearing the treatment device 192 and sleeping comfortably on the left side, according to some implementations of the present disclosure. FIG. 18B is a side view of the user 410 wearing the treatment device 192 of FIG. 15 and sleeping comfortably on the left side, according to some implementations of the present disclosure. Even though the convex surface 1504 does not rest along the pillow 1434, the treatment device 192 does not cause any discomfort to the user 410.

One or more elements or aspects or steps, or any portion(s) thereof, from one or more of any of claims 1-77 below can be combined with one or more elements or aspects or steps, or any portion(s) thereof, from one or more of any of the other claims 1-77 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for determining a percentage likelihood that a user has an untreated sleep disorder, the method comprising:
receiving first physiological data associated with the user during a first time period;
analyzing the first physiological data to determine (i) a first respiration rate for the first time period, (ii) a first plurality of sample heart rate values, and (iii) first heart rate variability parameters for the first time period;
receiving second physiological data associated with the user during a second time period;
analyzing the second physiological data to determine (i) a second respiration rate for the second time period, (ii) a second plurality of sample heart rate values, and (iii) second heart rate variability parameters for the second time period, the second respiration rate being less than the first respiration rate; and
determining the percentage likelihood that the user has an untreated sleep disorder based at least in part on the first heart rate variability parameters and the second heart rate variability parameters.

2. The method of claim 1, wherein the second respiration rate is at least 10% less, at least 20% less, at least 30% less, at least 40% less, at least 50% less, at least 60% less, or at least 70% less than the first respiration rate.

3. The method of claim 1, wherein the first heart rate variability parameters for the first time period include a maximum heart rate for the first time period, a minimum heart rate for the first time period, a heart rate range defined by the maximum heart rate and the minimum heart rate for the first time period, an average heart rate for the first time period, a median heart rate for the first time period, a standard deviation of heart rates for the first time period, or any combination thereof.

4. The method of claim 3, wherein the second heart rate variability parameters for the second time period include a maximum heart rate for the second time period, a minimum heart rate for the second time period, a heart rate range defined by the maximum heart rate and the minimum heart rate for the second time period, an average heart rate for the second time period, a median heart rate for the second time period, a standard deviation of heart rates for the second time period, or any combination thereof.

5. The method of claim 4, further comprising in response to the heart rate range for the second time period being no greater than the heart rate range for the first time period, determining that the percentage likelihood that the user has an untreated sleep disorder is greater than 40%, 50%, 60%, 70%, 80%, or 90%.

6. The method of claim 4, further comprising based at least in part on the heart rate range for the second time period not exceeding a threshold value, determining that the user is likely to have an untreated sleep disorder.

7. The method of claim 6, wherein the threshold value is 6 bpm, 7 bpm, 8 bpm, 9 bpm, 10 bpm, 11 bpm, 12 bpm, 13 bpm, 14 bpm, 15 bpm, 16 bpm, 17 bpm, 18 bpm, 19 bpm, 20 bpm, 21 bpm, or 22 bpm.

8. The method of claim 1, wherein the determining the percentage likelihood that the user has an untreated sleep disorder includes comparing the first heart rate variability parameters to the second heart rate variability parameters.

9. The method of claim 6, further comprising:
receiving a first time stamp associated with the first time period and a second time stamp associated with the second time period; and
based at least in part on the first time stamp and the second time stamp, determining the percentage likelihood that the user has an untreated sleep disorder.

10. The method of claim 6, further comprising:
receiving a first time stamp associated with the first time period and a second time stamp associated with the second time period; and
based at least in part on the first time stamp and the second time stamp, determining a severity of the untreated sleep disorder.

11. The method of claim 1, further comprising:
receiving additional physiological data associated with the user during an additional time period;
analyzing the additional physiological data to determine (i) an additional respiration rate for the additional time period, (ii) an additional plurality of sample heart rate values, and (iii) additional heart rate variability parameters for the additional time period, the additional respiration rate being less than the first respiration rate; and
determining a severity of the untreated sleep disorder based at least in part on the first heart rate variability parameters, the second heart rate variability parameters, and the additional heart rate variability parameters.

12. The method of claim 1, wherein the untreated sleep disorder includes untreated obstructive sleep apnea.

13. The method of claim 1, wherein (i) the first physiological data, (ii) the second physiological data, or (iii) both the first physiological data and the second physiological data are received from a mobile device coupled to the user's chest, a heart rate sensor, a pulse sensor, or any combination thereof.

14. The method of claim 13, wherein (i) the first physiological data, (ii) the second physiological data, or (iii) both the first physiological data are received from an accelerometer of the mobile device.

15. The method of claim 13, wherein (i) the first physiological data, (ii) the second physiological data, or (iii) both the first physiological data are received from a pulse oximeter, an ECG device, or both.

16. The method of claim 1, further comprising displaying an indication of the determined percentage likelihood that the user has an untreated sleep disorder.

17. The method of claim 16, further comprising in response to the determined percentage likelihood that the user has an untreated sleep disorder exceeding 50%, displaying the indication that the user is likely to have an untreated sleep disorder.

18. The method of claim 1, wherein (i) the first time period, (ii) the second time period, or (iii) both the first time period and the second time period are within 30 minutes, an hour, two hours, or three hours of the user waking up.

19. The method of claim 1, further comprising prior to receiving the second physiological data associated with the user during the second time period, causing the user to breathe slower than the first respiration rate.

20. The method of claim 19, wherein the causing the user to breathe slower than the first respiration rate includes instructing the user to stay still and relax.

21. The method of claim 1, further comprising:
receiving positional data associated with the user;
analyzing the received positional data to determine a body position of the user; and
based at least in part on the determined body position of the user and the determined percentage likelihood that the user has an untreated sleep disorder, causing the user to change body position.

22. The method of claim 21, wherein the causing the user to change the body position includes causing a sound or a vibration to be communicated to the user.

23. The method of claim 22, wherein a level of the sound or the vibration to be communicated to the user is (i) proportional to a determined severity of the untreated sleep disorder or (ii) gradually increased to awaken the user.

24. The method of claim 1, further comprising based at least in part on the determined percentage likelihood that the user has an untreated sleep disorder, instructing the user to wear a treatment device.

25. The method of claim 24, wherein the treatment device is configured to (i) generate sensor data, (ii) cause a sound or a vibration to be communicated to the user, or (iii) both (i) and (ii).

26. The method of claim 25, wherein the sensor data includes positional data associated with the user.

27. The method of claim 25, wherein the sensor data is generated using a motion sensor.

28. The method of claim 27, wherein the motion sensor includes an accelerometer, a gyroscope, a magnetometer, or any combination thereof.

29. The method of claim 25, further comprising analyzing the generated sensor data to determine (i) a sleep disorder event associated with the user, (ii) chest movement of the user, (iii) a heart rate of the user, or (iv) any combination thereof.

30. The method of claim 24, wherein the treatment device is a respiratory therapy system.

31. The method of claim 1, wherein the first respiration rate is more than six breaths per minute, and the second respiration rate is six or fewer breaths per minute.

32-42. (canceled)

43. A system comprising:
a control system including one or more processors; and
a memory having stored thereon machine readable instructions;
wherein the control system is coupled to the memory, and the method of claim 1 is implemented when the machine executable instructions in the memory are executed by at least one of the one or more processors of the control system.

44. A system for determining a percentage likelihood that a user has an untreated sleep disorder, the system including a control system configured to implement the method of claim 1.

45. (canceled)

46. A non-transitory computer readable medium comprising instructions which, when executed by a computer, cause the computer to carry out the method of claim 1.

47-77. (canceled)
